(19) United States
(12) Patent Application Publication
(10) Pub. No.: US 2026/0234565 A1
(43) Pub. Date: Aug. 13, 2026

(54) CELL POPULATIONS IN THE ANORECTAL TRANSITION ZONE WITH TISSUE REGENERATIVE CAPACITY, AND METHODS FOR ISOLATION AND USE THEREOF

(71) Applicant: Curileum Discovery Ltd, Harrow (GB)

(72) Inventors: Jeffrey G. Moore, London (GB); Priti Kalra, London (GB)

(21) Appl. No.: 19/156,555

(22) PCT Filed: Feb. 15, 2024
(86) PCT No.: PCT/GB2024/050416

§ 371 Date: Aug. 14, 2025

Related U.S. Application Data
(60) Provisional application No. 63/485,520, filed on Feb. 16, 2023.

(51) Int. Cl. C12N 5/071; A61K 35/38; A61P 13/12; C12N 5/077

(52) U.S. Cl. CPC C12N 5/068; A61K 35/38; A61P 13/12; C12N 5/0655; C12N 5/0657; C12N 5/0658; C12N 5/0686; C12N 2506/23

(57) ABSTRACT

Provided herein are compositions of anorectal transition zone stem cells, including multipotent and progenitor cells, to treat mesodermal disease. Also provided are cellular compositions of the anorectal transition zone stem cells, including multipotent and progenitor cells, in a pharmaceutically acceptable carrier.

### BACKGROUND

Transition zones of the gastrointestinal tissues exist between two different epithelial tissue types. Anorectal transition zone tissue is at the junction of rectal columnar epithelium of endodermal embryological origin and the anal stratified squamous, non-keratinised epithelium of ectodermal embryological origin (McNairn and Guasch (2011), “Epithelial transition zones: merging microenvironments, niches, and cellular transformation,” Eur J Dermatol, 21 (Suppl 2): 21-8). Histopathological labeling studies of mouse and human anorectal transition zone tissue have identified cells expressing p63, a marker for basal cells; cytokeratin 7 (CK7), a marker for simple columnar cells (Yang et al. (2015), “Microanatomy of the cervical and anorectal squamocolumnar junctions: a proposed model for anatomical differences in HPV related cancer risk,” Mod Pathol, 28 (7): 994-1000); and CD34, a marker for stem cells and progenitor cells (McNairn, supra). Further studies in mice have supported the presence of stem cells and progenitor cells in the anorectal transition zone using a label-retaining cell assay and immunohistochemistry confirming expression of p63, CK7, CD34, and identifying expression of SOX2, a pluripotent stem cell marker (Runck et al. (2010), “Identification of epithelial label-retaining cells at the transition between the anal canal and the rectum in mice,” Cell Cycle, 9 (15): 3039-45).

Stem cell therapy offers promise to repair mesodermal-derived tissues and organs that are dysfunctional or damaged by infection, medications, or physical trauma. Each of the current cell therapy sources has limitations in their utilities.

Human embryonic stem cells (ESCs) are pluripotent, giving rise to all cells in the body. They have regenerative potential but pose risks for teratoma formation and eliciting autoimmune responses. Encapsulating ESCs mitigates some of these risks. iPSCs, fibroblasts reprogrammed to differentiated cells, have mutagenic potential due to reprogramming methods, and have barriers to long-term transplant viability and functionality. Mesenchymal stem cells (MSCs) are widely used for their inherent anti-inflammatory properties, which reduce the risks of autoimmune responses and tumourigenicity. However, since MSCs are not regenerative, their benefit is transitory.

### SUMMARY

The invention is based, in part, upon the discovery of compositions for use (or suitable for use) in treating a tissue or organ derived from the mesoderm in a subject in need thereof, methods of making stem cell compositions useful in the treatment of a mesodermal tissue or organ, and methods of treating a mesodermal tissue or organ with such a stem cell composition.

In one aspect, provided herein are stem cell compositions for treating a mesodermal tissue or organ, the composition comprising isolated anorectal transition zone (ATZ) cells and/or ATZ-derived organoid cells. The isolated ATZ cells preferably include isolated ATZ crypt cells, including ATZ multipotent stem cells. The ATZ-derived organoid cells preferably include ATZ multipotent stem cells and ATZ-derived mesoderm cells which can be single cells, clusters of cells, 2-dimensional layers of cells, or 3-dimensional organoids. The cells can be cultured in suspension, adherent layers or liquid cultures. The compositions may comprise multipotent ATZ stem cells and mesodermal progenitor ATZ cells.

The isolated ATZ crypt cells comprise multipotent stem cells and/or progenitor cells that express KIT, LGR5, NANOG, and OCT4A. The multipotent isolated ATZ crypt cells can also express one or more additional stem cell and progenitor cell markers selected from the group consisting of BMP4, CD34, CXCR4, and SOX17. In some embodiments, the multipotent ATZ crypt cells express at least two, three or four stem cell and progenitor cell markers selected from the group consisting of BMP4, CD34, CXCR4, and SOX17.

The ATZ-derived organoids can express additional mesoderm markers depending upon the specific mesoderm organoid growth medium (LSM) used.

For example, in some embodiments, the ATZ-derived crypt cells are grown in a pluripotent stem cell MGM (e.g., mTesR™, STEMCELL Technologies, Inc, Vancouver, CA; StemFit® Feeder-Free Stem Cell Culture Media, AMSBIO, Abingdon, UK).

For example, in some embodiments, the ATZ-derived crypts cells were grown in an LSM to promote ventricular cardiomyocytes (e.g., STEMdiff™ Ventricular Cardiomyocyte Differentiation Kit, STEMCELL Technologies; PSC Cardiomyocyte Differentiation Kit, Thermo Fisher Scientific) and expressed one or more gene markers selected from the group: ISL1, KLF4, MEF2C, NKX2.5, PDGFRA, PECAM.

For example, in some embodiments, the ATZ-derived crypts cells were grown in a medium to promote multinucleated myotubes (e.g., MyoCult™, STEMCELL Technologies; Skeletal Muscle Differentiation Kit, AMSBIO, Abingdon, UK) and can express one or more gene markers selected from the group: ACTA1, DMD, MYH1, MYH2, MYOD1, MYOG, TNNT1, TNNT2.

For example, in some embodiments, the ATZ-derived crypts cells were grown in a medium to promote chondrocytes (e.g., MyoCult™, StemPro™ Chondrogenesis Differentiation Kit, Thermo Fisher Scientific) and can express one or more gene markers selected from the group: ACAN, COL2A1, GDF5, NOTCH (1-4), PRG4, SOX9.

The isolated ATZ crypt cells or ATZ-derived organoids do not express CD45 and, therefore, they are not of hematopoietic lineage.

In some embodiments, the ATZ-derived crypt cells grown in an intestinal-specific LSM (e.g., IntestiCult™; Human Intestinal Organoid Culture Protocol, Bio-Techne, Minneapolis, Minnesota) can, in addition, express ALP.

In some embodiments, the ATZ-derived crypt cells grown in a kidney-specific LSM (e.g., StemDiff Kidney™ Organoid Kit) can express one or more additional markers selected from the group consisting of EYA1, HAND1, LHX1, NPHS1, NPHS2, PAX2, SALL1 and SIX2. In some embodiments, ATZ-derived crypt cells grown in a kidney-specific LSM can express two, three, four, five, six or seven or eight additional markers selected from the group consisting of EYA1, HAND1, LHX1, NPHS1, NPHS2, PAX2, SALL1 and SIX2.

In some embodiments, the ATZ-derived crypts cells grown in a medium to promote ventricular cardiomyocytes (e.g., STEMdiff™ Ventricular Cardiomyocyte Differentiation Kit) can express one or more additional markers selected from the group: consisting of ISL1, KLF4, MEF2C, NKX2.5, PDGFRA and PECAM. In some embodiments, ATZ-derived crypt cells grown in a medium to promote ventricular cardiomyocytes can express two, three, four, five or six additional markers selected from the group consisting of ISL1, KLF4, MEF2C, NKX2.5, PDGFRA and PECAM.

In some embodiments, the ATZ-derived crypts cells grown in a medium to promote multinucleated myotubes (e.g., MyoCult™) can express one or more additional markers selected from the group consisting of ACTA1, DMD, MYH1, MYH2, MYOD1, MYOG, TNNT1 and TNNT2. In some embodiments, ATZ-derived crypt cells grown in a medium to promote multinucleated myotubes can express two, three, four, five, six, seven or eight additional markers selected from the group consisting of ACTA1, DMD, MYH1, MYH2, MYOD1, MYOG, TNNT1 and TNNT2.

The isolated ATZ crypt cells or ATZ-derived organoids can be combined with an exogenous biocompatible scaffold, for example, a synthetic scaffold or a biological scaffold. Depending upon the circumstances, the scaffold can comprise a collagen-based scaffold. Alternatively, the isolated ATZ crypt cells or ATZ-derived organoid cells can be combined with a matrix material (e.g., a gel) or a pharmaceutically acceptable carrier (e.g., saline).

The isolated ATZ crypt cells or ATZ-derived organoids can be porcine cells or human cells.

The isolated ATZ crypt cells or ATZ-derived organoid can be allogeneic or autologous to the subject in need of treatment.

The isolated ATZ crypt cells or ATZ-derived organoid can be genetically-modified to include selectable markers (e.g., to detect engraftment), to eliminate the cells (e.g., a suicide gene) or to reduce a host-versus-graft response (e.g., MHC or B2M knock-outs).

In certain embodiments, the composition is cryopreserved. To facilitate the cryopreservation, the composition may comprise a suitable cryopreservation media.

In another aspect, the invention provides methods of preparing the pharmaceutical compositions (e.g., any of the cellular compositions as disclosed herein). The methods comprise (a) harvesting ATZ tissue from a donor (e.g., a porcine donor or a human donor); (b) enzymatically digesting the tissue to prepare a cell suspension; (c) optionally combining at least a portion of the cell suspension with media for cryopreservation media and cryopreserving the cell suspension; and (d) combining the cell suspension of step (b) or the thawed suspension of optional step (c) with an exogenous biocompatible scaffold, matrix, or pharmaceutically acceptable carrier.

The cell suspension comprises isolated ATZ crypt cells and/or ATZ-derived organoid cells, which include multipotent stem cells. The ATZ crypt cells and/or ATZ-derived organoid cells, can also include mesoderm progenitor cells. In certain embodiments, the ATZ cells can differentiate into mesoderm cells (e.g., that express TBXT).

In some embodiments, the ATZ stem cells express CD34, CD117 (KIT), and CD184 (CXCR4), for example, as detected by flow cytometry. In addition, the ATZ stem cells do not express detectable levels of CD45, for example, as detected by flow cytometry. Furthermore, NANOG and/or OCT4A can be detected on freshly isolated ATZ crypt cells ATZ-derived organoids.

In certain embodiments, the cells in the suspension (e.g., ATZ stem cells) are expanded in vitro.

In certain embodiments, in step (d) of the method described above, cells in the cell suspension are combined with an exogenous biocompatible scaffold, for example, a synthetic scaffold or a biological scaffold. In certain embodiments, the scaffold comprises a collagen-based scaffold. Alternatively, the isolated ATZ crypt cells or ATZ-derived organoid cells can be combined with a matrix material (e.g., a gel) or a pharmaceutically acceptable carrier (e.g., saline).

In another aspect, the invention provides cellular compositions produced by the methods disclosed herein.

In another aspect, the invention provides methods for treating damaged or dysfunctional kidney tissue in a subject in need thereof, the methods comprising administering into the damaged or dysfunctional kidney tissue an effective amount of a cellular composition disclosed herein, thereby to treat the damaged or dysfunctional kidney tissue.

In another aspect, the invention provides methods for treating damaged or dysfunctional heart tissue in a subject in need thereof, the methods comprising administering into the damaged or dysfunctional heart tissue an effective amount of a cellular composition disclosed herein, thereby to treat the damaged or dysfunctional heart tissue.

In another aspect, the invention provides methods for treating damaged or dysfunctional striated muscle tissue in a subject in need thereof, the methods comprising administering into the damaged or dysfunctional striated muscle tissue an effective amount of a cellular composition disclosed herein, thereby to treat the damaged or dysfunctional striated muscle tissue.

In another aspect, the invention provides methods for treating damaged or dysfunctional cartilage in a subject in need thereof, the methods comprising administering into the damaged or dysfunctional cartilage an effective amount of a cellular composition disclosed herein, thereby to treat the damaged or dysfunctional cartilage.

### BRIEF DESCRIPTION OF THE DRAWINGS

FIG. 1 shows isolation of ATZ crypts from porcine tissue: (a) shows resected colorectum tissue; (b) shows exposed mucosal epithelium, (c) shows excised dentate line, (d) shows freshly isolated ATZ crypts, (e) shows freshly isolated ATZ crypts, (f) shows freshly isolated ATZ crypts, (g) shows freshly isolated bifurcating ATZ crypt, (h) shows freshly isolated ATZ submucosal gland, and (i) shows freshly isolated ATZ submucosal gland.

FIG. 2 shows isolation of ATZ crypts from Crohn's disease patient tissue: (a), shows resected anorectum tissue, (b) shows excised mucosal epithelium, (c) shows brightfield images of freshly isolated ATZ crypts, and (d) shows brightfield images of freshly isolated ATZ crypts.

FIG. 3 shows (a) crypt organoid development in porcine GI tissue, and (b) crypt organoid development in Crohn's disease patient rectum and ATZ tissue.

FIG. 4 shows increased progenitor capacity of single cell preparations of porcine ATZ crypts as compared to rectal crypts. FIG. 4(a) shows porcine ATZ and rectal crypt growth over 4 weeks. FIG. 4(b) shows increased plating efficiencies over two months of crypt organoids derived from ATZ cells compared to rectal cells. Cells were isolated and replated after 1 month.

FIG. 5 shows protein expression of stem cell and progenitor cell markers on freshly isolated crypts and crypt organoids derived from pigs and a Crohn's disease patient: (a) shows CD117 expression on crypt-derived single cells from porcine GI tissues, (b) shows CD34 expression on porcine fresh crypt-derived single cells from porcine GI tissues, (c) shows double labelling by flow cytometry of CD34 and CD117 expression on fresh porcine ATZ crypt cells, (d) shows stem cell and progenitor marker expression on porcine crypt organoids (Small intestine is shown on the left bar, rectum is shown in the middle bar, ATZ is shown on the right bar), (e) shows the indicated developmental lineage marker expression on fresh porcine ATZ crypt-derived cells, (f) shows stem cell and progenitor marker expression on Crohn's disease patient rectal crypt-derived organoids.

FIG. 6 shows mRNA profiling from freshly isolated crypts and crypt organoids of the porcine ATZ: (a) mRNA profiling of freshly isolated porcine ATZ crypt cells, (b) shows mRNA profiling of organoids derived from porcine ATZ crypt cells.

FIG. 7 shows flow cytometric analysis of stem cell and progenitor cell markers to assess growth media at day 14 to promote porcine ATZ crypt stem cell expansion, wherein (a) shows CD117 expression, (b) shows Brachyury expression, and (c) shows SOX17 expression.

FIG. 8 shows that porcine ATZ crypt cells generate mature cells of the endoderm lineage: (a)-(d) shows brightfield images of organoid development from days 1, 4, 7, and 12, respectively, (e)-(h) shows DAPI staining of nuclei and immunocytochemistry images of organoids, wherein, (e) shows lysosome expression, (f) shows Ki67 expression, (g) shows Muc-2 expression, (h) shows CK18 expression.

FIG. 9 shows flow cytometry analysis of endodermal markers on ATZ organoids at days 0, 7, and 14.

FIG. 10 shows that porcine ATZ crypt cells generate mature cells of the mesoderm lineage: (a)-(d) shows brightfield images of blood vessel-like development, wherein (a) shows undifferentiated ATZ single cells at day 0, (b) shows ATZ single cells starting to form clusters at day 3, (c) shows blood vessel-like structures starting to form at day 8, (d) shows a network of blood vessel-like structures forming at day 10. (e) shows DAPI stained nucleus and immunocytochemistry of CD31 expressing endothelial-like cells, and (f) shows DAPI stained nucleus and immunocytochemistry of CD31 expressing blood vessel-like cells.

FIG. 11 shows that porcine ATZ crypt cells generate mature cells of the mesoderm lineage as detected by flow cytometry for mesodermal markers, brachyury and CD31, on ATZ organoids at days 0, 7, and 14.

FIG. 12 shows porcine ATZ crypt cells generate mature cells of the ectoderm lineage: (a) shows brightfield images of keratinocyte development from anal skin and ATZ crypts grown in KFSM, (b) shows immunocytochemistry of K14 and K15 markers on anal skin cultures, (c) shows immunocytochemistry of K14 and K15 markers on ATZ crypt cultures, (d) shows flow cytometry analysis of ectodermal markers PAX6, NESTIN, and CK14 on ATZ cultured cells on days 0, 7 and 14.

FIG. 13 shows brightfield images of porcine small intestine crypt single cells in medium to promote differentiation in the 3 developmental lineages at days 0, 7, and 14, wherein (a) shows culture in human endodermal medium (IntestiCult™), (b) shows culture in human mesodermal medium (MethoCult™), (c) shows culture in ectodermal medium (KFSM).

FIG. 14 shows the results of an in vitro embryoid body assay to test pluripotency of porcine ATZ crypt single cells: (a) shows brightfield images of ATZ embryoid body development, (b) shows alkaline phosphatase staining of ATZ embryoid bodies, (c) shows immunostaining of ATZ embryoid bodies for expression of pluripotent stem cell markers SSEA4 and OCT4, (d) shows immunostaining of ATZ embryoid bodies with pluripotent stem cell markers SOX2 and TRA-1-60 expression. DAPI was used for nucleus staining.

FIG. 15 shows isolation of stem cells and progenitor cells from porcine kidney tissue: (a) shows resected kidney tissue, (b) shows diced kidney tissue after enzymatic treatment, (c) shows freshly isolated kidney stem cells and progenitor cells.

FIG. 16 shows culture of porcine ATZ crypts and kidney stem cells and progenitors in kidney LSM after one passage: (a) kidney organoid-like structures derived from ATZ crypts, (b) kidney organoids derived from kidney stem cells and progenitors.

FIG. 17 shows cardiomyocyte-like organoids derived from porcine ATZ crypts cultured in cardiomyocyte LSM for after (a) 3 days and (b) 1 day after passage.

FIG. 18 shows myocytes and multinucleated myotube-like structures derived from porcine ATZ crypts cultured in myocyte LSM for after (a) 19 days and (b) 26 days.

FIG. 19 shows developing chondrocytes derived from porcine ATZ crypts cultured in chondrocyte LSM for after (a) 1 day and (b) 4 days.

FIG. 20 shows the isolation of ATZ crypts from an idiopathic perianal fistula patient: (a) biopsy taken from anorectal tissue, (b) brightfield images of freshly isolated ATZ crypts, and (c) brightfield images of freshly isolated ATZ single cell suspension.

FIG. 21(a-b) shows mRNA expression by qPCR of freshly isolated ATZ crypts of two idiopathic fistula perianal patients. Ct values are normalized to housekeeping gene GAPDH.

FIG. 22 shows organoid development in IntestiCult™ from a single cell suspension generated from ATZ crypts of idiopathic perianal fistula patient. Clusters of cells formed on day 4 and organized structures formed on day 7 (both at passage 0); compact, thickened organoids formed on day 7 and day 14 of passage 1.

FIG. 23 shows formation of kidney organoids derived from idiopathic perianal fistula patient ATZ cells and cultured in STEMdiff™ Kidney Organoid Kit.

### DETAILED DESCRIPTION

### Definitions

As used herein, the following terms and phrases shall have the meanings set forth below. Unless defined otherwise, all technical and scientific terms used herein have the same meaning as commonly understood by one of ordinary skill in the art to which this disclosure belongs.

As used herein, the term “gastrointestinal mucosa” or “gastrointestinal mucosal epithelium” refers to the epithelial mucosal layers of the gastrointestinal tract including the mouth, pharynx (throat), oesophagus, stomach, small intestine, large intestine, rectum, and anus. The “gastrointestinal mucosa” or “gastrointestinal mucosal epithelium” includes the oral, pharyngeal, oesophageal, gastric, intestinal, rectal and anal mucosa or mucosal epithelium.

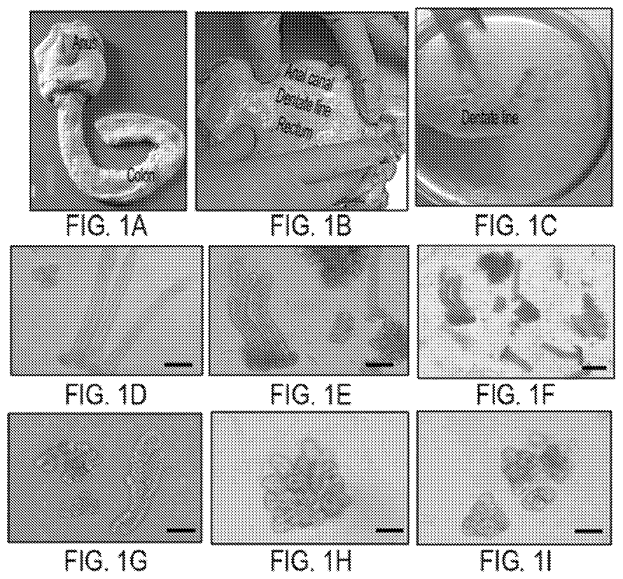

As used herein, the term “anorectal transition zone” or “ATZ” refers to the intestinal epithelium interposed between the uninterrupted squamous epithelium of the anoderm and dentate (or pectinate) line below and the uninterrupted rectal columnar epithelium above. The dentate line is the junction between the superior and inferior anal canal. There are many differences between these two regions, including their embryological origins, innervation, venous and arterial supply, and lymphatic supply. Above the dentate line, the epithelium of the anal canal has an endodermal origin and is lined by simple columnar epithelia. Below the dentate line, the epithelium of the anal canal has an ectodermal origin and is predominantly lined by stratified squamous epithelium. The epithelium of the ATZ is typically 1-4 mm wide and can easily be identified and biopsied by those skilled in the art (see, e.g., FIG. 1).

As used herein, the term “anorectal transition zone cells” or “ATZ cells” refers to a mixed population of cells derived from anorectal transition zone epithelial tissue comprising crypts, submucosal glands, and other epithelial cells.

As used herein, the term “isolated ATZ cells” refers to a mixed population of cells physically isolated (e.g., by biopsy sample) from the ATZ epithelium. Optionally, the ATZ tissue can be physically minced or enzymatically digested to isolate ATZ cells.

As used herein, the term “crypt cells” refers to the cells of the crypts of Lieberkühn, structures below the surface of the intestinal mucosal lining, and comprising stem cells that are responsible for continuously regenerating intestinal mucosa throughout life.

As used herein, “ATZ crypt cells” refers to the crypt cells of the ATZ, which include multipotent stem cells (i.e., cells that have the capacity to self-renew by dividing and to develop into multiple specialised cell types present in a specific tissue or organ), progenitor cells, and mature crypt cells. Multipotent ATZ cells are stem cells that have the capacity to differentiate into multiple somatic cell lineages including endodermal cells (e.g., intestinal mucosa), mesodermal cells (e.g., blood vessels), and/or ectodermal cells (e.g., skin).

As used herein, the term “isolated ATZ crypt cells” refers to a mixed population of cells (e.g., multipotent stem cells, progenitor cells, fully differentiated or mature cells) forming the crypts that have been physically isolated (e.g., by dissection) from the ATZ epithelium. Alternatively, or in addition, the ATZ crypts can be physically, chemically, or enzymatically dissociated to further isolate the ATZ crypt cells.

As used herein, the term “stem cells” refers to undifferentiated or partially differentiated cells that can differentiate into various types of cells and proliferate indefinitely to produce more of the same stem cell (i.e., self-renewing).

As used herein, “self-renewal” is the process by which stem cells divide to make more stem cells, perpetuating the stem cell pool throughout life. Self-renewal is division with maintenance of the undifferentiated state. This requires cell cycle control and often maintenance of multipotency or pluripotency, depending on the stem cell.

As used herein, the term “progenitor cells” refers to stem cells with the potential to differentiate into a single cell type or lineage, and the term “progenitor ATZ cells” means progenitor cells derived from the ATZ crypt cells.

As used herein, the term “multipotent cells” refers to stem cells with the potential to differentiate into at least two cell types or lineages, and the term “multipotent ATZ cells” means multipotent cells derived from the ATZ crypt cells.

As used herein, the term “pluripotent cells” refers to stem cells with the potential to differentiate into each of the three primary groups of cells, i.e., ectoderm, mesoderm and endoderm, and the term “pluripotent ATZ cells” means multipotent cells derived from the ATZ crypt cells.

As used herein, the term “isolated ATZ stem cells” refers to a mixed population of stem cells (e.g., pluripotent stem cells, multipotent stem cells, and progenitor cells) derived from the ATZ crypts that have been isolated (e.g., physically, chemically, or enzymatically dissociated) to further isolate the ATZ crypt cells from the ATZ crypts. Alternatively, or in addition, the ATZ stem cells can be numerically expanded relative to other ATZ cells by one or more rounds of replating/passaging because of their superior properties of stem cell self-renewal.

As used herein, the terms “proliferation” and “proliferating” refer to an increase in cell number by mitosis.

As used herein, the term “differentiation” refers to the formation of cells expressing markers known to be associated with cells that are more specialised and closer to becoming terminally differentiated cells incapable of further division or differentiation. For example, in a haematological context, differentiation can be seen in the production of functional cells of multiple cellular lineages (e.g., red blood cells, platelets, granulocytes, macrophages). The terms “further” or “greater” differentiation refers to cells that are more specialised and closer to becoming terminally differentiated cells incapable of further division or differentiation than the cells from which they were cultured.

As used herein, the term “terminally differentiated” refers to cells that are incapable of further differentiation and/or further division/proliferation or differentiation.

As used herein, the term “expanded” when referring to cells, means cells that have been increased in number by proliferation and differentiation in vitro.

As used herein, the term “minimal growth medium” or “MGM” refers to a cell culture medium which provides the nutrients necessary for ATZ-derived cell maintenance without promoting differentiation of multipotent ATZ-derived stem cells to an extent that results in a decreased absolute number of multipotent ATZ-derived stem cells.

As used herein, the term “lineage-specific medium” or “LSM” refers to a cell culture medium which provides the nutrients and growth factors necessary for multipotent ATZ-derived stems to undergo differentiation toward specific-lineage cells, and to result in a decreased absolute number of multipotent ATZ-derived stem cells. The lineage-specific differentiation may be partial (e.g., toward endoderm, mesoderm or ectoderm progenitors) or terminal (e.g., toward goblet cells, cardiac muscle, neurons).

As used herein, the term “eATZ stem cells” refers to an expanded population of ATZ stem cells that can be generated in suspension or adherent 2 D cultures (e.g., individual cells) (e.g., clusters) or 3D cultures consisting of complex structures (e.g., organoids).

The expanded population of cells may retain the ability to differentiate into one or more cell types. In some embodiments, the eATZ cells retain at least one marker of ATZ stem cells selected from the group consisting of CD34, CD117 (KIT), CD184 (CXCR4), OCT4, NANOG, SOX17, Brachyury (TBXT), PAX6 and NESTIN.

The expanded population of eATZ cells can be differentiated in LSM to generate a variety of organoids, including intestinal organoids or ATZ stem cell-derived pancreatic organoids or hepatic organoids or lung organoids for stem cell transplantation.

As used herein, the term “organoids” refers to a multiplicity of cells grown in culture which self-organize to form a three-dimensional structure. Organoid structures may be a simple solid mass of cells, a hollow mass (e.g., a cystic organoid), a tube, or a more complicated structure (e.g., a crypt-like or follicle-like structure).

As used herein, the term “cellular composition” refers to a preparation of cells, which preparation may include, in addition to the cells, non-cellular components such as cell culture media, e.g., proteins, amino acids, nucleic acids, nucleotides, co-enzyme, antioxidants, metals and the like. Furthermore, the cellular composition can have components which do not affect the growth or viability of the cellular component, but which are used to provide the cells in a particular format, e.g., as polymeric matrix for encapsulation or a pharmaceutical preparation.

As used herein, the term “phenotype” refers to the observable or detectable characteristics of a cell, such as size, morphology, RNA expression, protein expression, or other properties.

As used herein, the term “marker” refers to a biological molecule that can be used to identify the phenotype of a cell by its presence, absence, or concentration, or by its activity, inactivity, or level of activity.

As used herein, the term “exogenous biocompatible scaffolds” refers to three-dimensional porous, fibrous, or permeable volume-retaining biomaterials intended to provide physical or mechanical support while permitting diffusion or transport of body liquids and gases, allowing for cellular interactions. Preferably, such scaffolds cause limited or minimal inflammation and toxicity. Optionally, such scaffolds are biodegradable. Examples of scaffolds can include a biological scaffold (e.g., a laminin or collagen-based scaffold) and synthetic scaffolds (e.g., non-biological polymers such as PGA, PLA, PLGA). Scaffolds may be in the physical form of a thread, sheet, paste, powder, or liquid. Scaffolds can be used in vitro and in vivo.

As used herein, the phrase “pharmaceutically acceptable” refers to those compounds, materials, compositions, and/or dosage forms which are, within the scope of sound medical judgment, suitable for use in contact with the tissues of human beings and animals without excessive or unacceptable toxicity, irritation, allergic response, inflammatory response or other problem or complication, commensurate with a reasonable benefit/risk ratio.

As used herein, the phrase “pharmaceutically-acceptable carrier” means a pharmaceutically acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

As used herein, the term “chronic kidney disease” refers to several diseases including chronic kidney disease, diabetic nephropathy, polycystic kidney disease, and Alport syndrome.

As used herein, “significant levels” of a stem cell marker can be determined by standard quantitative methods which are suitable to the marker (e.g., quantitative detection of antibody or other ligand bidning to cell surface proteins; quantitative detection of mRNA expression by quantitative PCR; quantitative detection of catalytic products for emzymes). For each method, a standard error of measurement or a standard level of background noise can be determined. If the measured level of expression is not statistically significant at the 5% level for the chosen methods, then the marker is not expressed at “significant levels.”

As used herein, the terms “acute cardiac disease” refers to myocardial infarctions and acute heart failure.

As used herein, the terms “chronic cardiac disease” refers to coronary artery disease and congestive heart failure.

As used herein, the term “chronic muscle diseases” refers to several skeletal muscle diseases including muscular dystrophy, myasthenia gravis, polymyositis.

As used herein, the term “chondrocyte diseases” refers to several diseases, including osteoarthritis, rheumatoid arthritis, and chondrosarcoma.

### Multipotent and Progenitor Atz Cells and Organoid Compositions

The multipotent and/or progenitor ATZ cells described herein can be isolated as single cell (or multiple cell) preparations derived from primary ATZ tissue, including isolated ATZ crypt cells. The ATZ cells described herein, including multipotent and progenitor ATZ cells, can be isolated from ATZ crypts obtained by biopsy, or can be derived from organoid structures differentiated ex vivo from isolated ATZ crypt cells. In some embodiments, the multipotent and progenitor ATZ cells are obtained from organoids derived from single primary ATZ crypt cells or dissociated ATZ crypt cells.

For use in the methods of treatment described herein, the multipotent or progenitor ATZ cells are preferably autologous cells derived from a human patient or allogeneic cells derived from a human donor (preferably tissue-matched to reduce or avoid graft-versus-host or host-versus-graft immunoreactivity). Alternatively, the multipotent or progenitor ATZ cells can be derived from other mammalian species and can be modified by methods known in the art to reduce or eliminate alloreactivity (e.g., gene editing to knock-out MHC Class I and/or Class II genes and/or the 32-microglobulin gene).

In some embodiments, the multipotent or progenitor ATZ cells can be in vitro expanded ATZ (eATZ) cells.

In some embodiments, the multipotent and progenitor ATZ cells, primary ATZ cells, isolated ATZ crypt cells and/or ATZ-derived organoids are combined in vivo or in vitro with an exogenous biocompatible scaffold, for example, a synthetic scaffold or a biological scaffold. In some embodiments, the scaffold comprises laminin and/or collagen (e.g., Permacol™ Paste, Medtronic PLC, Minneapolis, MN). In some embodiments, the scaffold is Corning® Matrigel® Matrix (Corning, Inc, New York) or a functional equivalent.

In some embodiments, the scaffold comprises a functionalised collagen scaffold or a functional equivalent.

The exogenous biocompatible scaffold can also comprise cell culture medium components. In some embodiments, the scaffold comprises a mesodermal lineage-specific medium (LSM), which can be purchased from commercial vendors (e.g., StemDiff Kidney™ Organoid Kit, STEMdiff™ Ventricular Cardiomyocyte Differentiation Kit, MyoCult™, DiffMesenCult™-ACF Chondrogenic Differentiation Kit, Vancouver, CA).

In some embodiments, the ATZ cells or ATZ crypt cells can be cultured in an MGM. In some embodiments, the ATZ cells or ATZ crypt cells can be cultured in a feeder-free medium that maintains human embryonic stem cells (ESCs) and/or induced pluripotent stem cells (iPSCs) in an undifferentiated state (e.g., mTesR™, STEMCELL Technologies). In some embodiments, the scaffold comprises such an ESC or iPSC feeder-free medium. In some embodiments, the ATZ cells or ATZ crypt cells cultured in LSM have a higher expression of mesoderm and endoderm lineage markers as compared to ATZ cells or ATZ crypt cells cultured in such an ESC or iPSC feeder-free medium. The matrix can also comprise keratinocyte serum-free medium (KSFM) for differentiation. Such KSFM medium is commercially available from vendors (e.g., Gibco™Keratinocyte-SFM Medium, Thermo Fisher Scientific; DermaCult™ Keratinocyte Expansion Medium, STEMCELL Technologies).

In another aspect, the disclosure provides for phenotypic characterisation of extracellular and intracellular protein and mRNA expression levels of stem cell and progenitor cell markers on freshly isolated ATZ cells. ATZ cells can express at least one of CD34, CD117, and CD184 cell surface markers but do not express detectable levels of CD45 as detected by flow cytometry. In some embodiments, isolated porcine ATZ cells express OCT4 and NANOG.

ATZ stem cells can also express markers for stem cells of three developmental lineages: endoderm (SOX17), mesoderm (TBXT) and ectoderm (PAX6 and NESTIN). In addition, freshly isolated ATZ cells can express markers for pluripotent stem cells (NANOG and OCT4).

In some embodiments, the ATZ cells and/or crypts express CD34. In some embodiments, the ATZ cells and/or crypts do not express detectable levels of CD45. In some embodiments, the ATZ cells and/or crypts express CD34 and do not express detectable levels of CD45.

In some embodiments, the ATZ cells and/or crypts express at least one marker selected from the group consisting of ALP, TBXT, BMP4, CD34, KIT, CXCR4, CHGA, CK18, EPCAM, LGR5, LYS, MUC2, NANOG, PAX6, SOX17, and OCT4. In some embodiments, the ATZ cells and/or crypts express at least 2, 3, 4, 5, 6, 7, 8, 9, 10, 11, 12, 13, 14, 15 or 16 markers selected from the group consisting of ALP, TBXT, BMP4, CD34, KIT, CXCR4, CHGA, CK18, EPCAM, LGR5, LYS, MUC2, NANOG, PAX6, SOX17, and OCT4.

In some embodiments, the ATZ cells and/or crypts express at least one marker selected from the group consisting of ALP, TBXT, BMP4, CD34, KIT, CXCR4, CHGA, CK18, EPCAM, LGR5, LYS, MUC2, PAX6, and SOX17. In some embodiments, the ATZ cells and/or crypts express at least 2, 3, 4, 5, 6, 7, 8, 9, 10, 11, 12, 13 or 14 markers selected from the group consisting of ALP, TBXT, BMP4, CD34, KIT, CXCR4, CHGA, CK18, EPCAM, LGR5, LYS, MUC2, PAX6 and SOX17. In some embodiments, the ATZ cells and/or crypts express OCT4 or NANOG genes.

In some embodiments, the ATZ cells and/or crypts express CD117 (KIT) at a higher level as compared to small intestine, colon, and rectum crypt cells. In some embodiments, the ATZ cells and/or crypts express CD117 (KIT) at least 1.5-fold, 2-fold, 2.5-fold, 3-fold, 3.5-fold, 4-fold, 4.5-fold, 5-fold, 5.5-fold, 6-fold, 6.5-fold, 7-fold or higher as compared to small intestine, colon, and rectum crypt cells.

An aspect of the disclosure provides for phenotypic characterisation of extracellular and intracellular protein and mRNA expression levels of stem cell and progenitor cell markers on ATZ organoids in culture. In some embodiments, the ATZ organoids express genes associated with endoderm, ectoderm, and mesoderm lineages. In some embodiments, the ATZ organoids express at least one protein marker selected from the group consisting of alkaline phosphatase, brachyury, CD34, CD117 (KIT), chromogranin, cytokeratin 18, CD184 (CXCR4), EpCAM, GD2, LGR5, lysozyme, mucin 2, Nestin, and PAX6. In some embodiments, the ATZ organoids express CD34. In some embodiments, the ATZ organoids do not express detectable levels of CD45. In some embodiments, the ATZ organoids express at least one gene selected from the group consisting of ALP, TBXT, BMP4, CD34, KIT, CXCR4, CHGA, CK18, EPCAM, LGR5, LYS, MUC2, NANOG, PAX6, and SOX17.

In some embodiments, the ATZ organoids express at least one cell surface marker selected from the group consisting of CD31, CD34, CD117 (KIT), CXCR4 (CD184), CK14, CK15, CK18 GD2, SOX2, SSEA4, and TRA-1-60. In some embodiments, the ATZ organoids express CD34. In some embodiments, the ATZ organoids do not express detectable levels of CD45. In some embodiments, the ATZ organoids express CD34 but do not express detectable levels of CD45.

In some embodiments, the ATZ organoids express CD117 (KIT), CXCR4 (CD184), and/or GD2 at a higher level as compared to small intestine and rectum organoids. In some embodiments, the ATZ organoids express CD117 (KIT) at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids express CXCR4 (CD184) at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids express GD2 at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids have reduced SOX17 expression as compared to ATZ crypt cells.

In some embodiments, the ATZ organoids have reduced TBXT expression as compared to ATZ crypt cells. In some embodiments, the ATZ organoids have reduced CD31 expression as compared to ATZ crypt cells. In some embodiments, the ATZ organoids have reduced PAX6 expression as compared to ATZ crypt cells. In some embodiments, the ATZ organoids have increased LGR5 as compared to ATZ crypt cells. In some embodiments, the ATZ organoids have increased CD117 or CXCR4 as compared to ATZ crypt cells. In some embodiments, the ATZ organoids have increased K14 as compared to ATZ crypt cells. In some embodiments, the ATZ organoids have increased expression of CD117, CXCR4, LGR5, or K14 of at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to ATZ crypt cells. In some embodiments, the ATZ organoids have decreased expression of CD31, or PAX6, SOX17, or TBXT of at least 1.5-fold, 2-fold, 2.5-fold, 3-fold, 3.5-fold, 4-fold, 4.5-fold, 5-fold, 5.5-fold, 6-fold, 6.5-fold, or 7-fold lower as compared to ATZ crypt cells.

Protein and nucleic acid expression can be determined by methods known to one of skill in the art. Such methods include, but are not limited to, polymerase chain reaction (PCR), RTPCR, q-RT-PCR, flow cytometry (combined with binding agents (e.g., labelled antibodies or ligands for cell surface markers), SDS-PAGE, mass spectrometry, immunoblotting (Western blotting), immunofluorescence microscopy, fluorescence in situ hybridisation, or any other technique known in the art.

In some embodiments, the ATZ organoids comprise proliferating cells, secretory cells, and cytokeratin. In some embodiments, the ATZ organoids can secrete lysozyme.

### Methods of Isolating and Producing Populations of Multipotent and Progenitor Atz Cells

In another aspect, provided herein are methods of isolating and producing multipotent and/or progenitor ATZ cells. The ATZ cells can be produced by obtaining anorectal transition zone tissue from a mammalian subject (e.g., human, porcine) either from biopsied or resected tissue; washing tissues with a serum-free tissue culture medium optionally containing antibiotics and/or antimycotics; mincing the tissue; and enzymatically treating or digesting the tissues with a collagenase. Digestion can be stopped by, for example, adding protein such as albumin. Crypts can be isolated by rigorously stirring or shaking the tissue digest to release the crypts. Medium containing crypts can be passed through tissue strainers to remove large debris and centrifuged to sediment or pellet crypts; and the crypts can be resuspended in fresh medium to obtain a population of multipotent and/or progenitor ATZ cells.

Resuspended crypts can be enumerated under a microscope. Single cell preparations from crypts are generated by physical disruption with a needle/syringe and/or treatment with mild enzymatic cell dissociation reagents to obtain a population of multipotent and/or progenitor ATZ cells. These cells can then be embedded in an in vitro matrix scaffold (for example, as described above), and cultured with differentiation media to produce organoids.

In some embodiments, the enzymatic treatment of minced ATZ tissue releases intestinal crypts and submucosal glands. In some embodiments, the anorectal transition zone tissue is from a healthy human donor. In some embodiments, the anorectal transition zone tissue is from a diseased human donor.

The released intestinal crypts or multipotent and/or progenitor ATZ cells can then be differentiated into organoids after being placed in a growth medium or a scaffold (e.g., laminin, collagen, Permacol™, Corning® Matrigel® Matrix). Any suitable matrix and growth medium know in the art can be used. In some embodiments, the crypts are placed in a synthetic scaffold. In some embodiments, the scaffold is non-functionalised.

In some embodiments, the scaffold is functionalised. Alternatively, the crypts can be further dissociated into single ATZ cells.

In some embodiments, the isolated ATZ-derived crypt cells have increased multipotent and/or progenitor capacity as compared to cells derived from rectum crypts.

In another aspect, the invention provides for in vitro maintenance of ATZ stem cells (including multipotent and/or progenitor cells) by culturing crypts or single cells embedded in a scaffold (e.g., Permacol™, Corning® Matrigel® Matrix) with a minimal growth medium (MGM). The use of MGM permits maintenance of the percentage of multipotent ATZ stem cells (+/−10%) without promoting terminal differentiation the cells. To be clear, in a mixed culture of ATZ cells, including ATZ-derived crypt cells and/or ATZ-derived organoid cells, there will be multipotent ATZ stem cells, ATZ progenitor cells, and partially or terminally differentiated ATZ cells, and some cells will become more differentiated over time. However, if the rate of multipotent ATZ stem cell proliferation equals or exceeds the rate of differentiation (and loss of multipotency), the ATZ stem cell culture can be maintained indefinitely. If the rate of multipotent ATZ stem cell proliferation is slightly exceeded by the rate of differentiation (and loss of multipotency), the ATZ stem cell culture can nonetheless be maintained for several weeks, which is sufficient for the methods of treatment described herein.

Preferably, ATZ organoid cultures are refreshed on periodic basis by breaking-up organoids and replating the ATZ-derived organoid cells in MGM. Using an appropriate MGM, it is possible to maintain at least 5%, at least 10% or at least 15% multipotent ATZ stem cells after at least 3 weeks, 4 weeks, at least 5 weeks, or at least 6 weeks of organoid culture. For example, the methods described herein allow at least 5% multipotent stem cells to be obtained after 5 passages, 10% after 10 passages, and 10% after 20 passages. Percentages of multipotent ATZ stem cells are relative to total ATZ cells.

In another aspect, the invention provides for methods of in vitro expansion of ATZ stem cells (including multipotent and/or progenitor cells) in order to produce sufficiently large populations of cells (e.g., 10³, 10⁴, 10⁵, 10⁶, 10⁷, 10⁸) to be useful for cell transplantation into damaged or dysfunctional mesodermal tissues. The methods can include repeated passaging of ATZ organoids in MGM. To be clear, during expansion, the absolute number of multipotent ATZ-derived stem cells can increase while the percentage of multipotent ATZ-derived stem cells decreases. For purposes of transplantation into a damaged or dysfunctional tissue, the absolute number of cells will be more relevant in many applications. The methods of expansion can further include confirmation that harvested cells maintain desired phenotypic properties determined, for example, by protein and mRNA expression levels of cell surface and intracellular stem cell markers and replating efficiencies. Once the properties of ATZ cells are validated for a cell source, a biomarker such as a cell surface marker (e.g., CD34, CD117, and/or CD184) can be used to determine appropriate numbers of cells for stem cell therapy.

Examples of commercially available MGM include mTesR™ (STEMCELL Technologies, Inc, Vancouver, CA) and StemFit® Feeder-Free Stem Cell Culture Media (AMSBIO, Abingdon, UK). In addition, despite its name, IntestiCult™; Human Intestinal Organoid Culture Protocol (Bio-Techne, Minneapolis, MN) can be used as an MGM at lower concentrations because the rate of multipotent ATZ stem cell proliferation can equal or exceed the rate of differentiation (and loss of multipotency).

In some embodiments, a mixed culture of ATZ-derived stem cells and ATZ-derived organoids, including multipotent ATZ-derived stem cells, can be induced to differentiate toward mesodermal tissue by culturing the cells in a mesodermal lineage-specific medium (LSM). The resulting mixed population of cells will include a greater percentage of ATZ-derived mesodermal progenitor cells which can be used in the methods of treatment described herein.

Examples of commercially available mesoderm LSMs include STEMdiff™ Ventricular Cardiomyocyte Differentiation Kit (STEMCELL Technologies) and PSC Cardiomyocyte Differentiation Kit (Thermo Fisher Scientific) (ventricular cardiomyocytes); MyoCult™ (STEMCELL Technologies); Skeletal Muscle Differentiation Kit (AMSBIO, Abingdon, UK) (multinucleated myotubes); MyoCult™, StemPro™ Chondrogenesis Differentiation Kit (Thermo Fisher Scientific) (chondrocytes) and StemDiff Kidney™ Organoid Kit (kidney).

An aspect of the disclosure provides for cryopreservation and thawing of ATZ cells after harvesting ATZ crypts, single cells and/or after organoid culture by resuspending centrifuged cells in serum-free, DMSO-containing freezing medium and gradually decreasing the temperature to −80° C. or lower. To re-establish organoid cultures, cryopreserved ATZ organoids or single cells are quickly thawed in a 37° C. water bath, resuspended in pre-warmed tissue culture medium, and centrifuged and resuspended in fresh medium to remove freezing medium. In some embodiments, the composition further comprises cryopreservation media. In some embodiments, the ATZ or eATZ composition is cryopreserved.

In some embodiments, ATZ-derived mesoderm-derived organoids (e.g., kidney, heart, striated muscle, and cartilage) are generated from biopsies taken from a patient with an abnormal functioning tissue or organ.

### Methods of Treatment

In some embodiments, the ATZ crypts are cultured ex vivo to produce eATZ cells for use in a method of treating tissues and organs of mesodermal origin. In some embodiments, the eATZ cells can be cryopreserved after expansion and then thawed prior to treatment.

In some embodiments, the pharmaceutically acceptable carrier comprises an exogenous biocompatible scaffold, for example, a scaffold described hereinabove.

In another aspect, provided herein are methods of preparing a therapeutic or pharmaceutical composition (e.g., a cellular composition as disclosed herein). The method comprises (a) harvesting ATZ tissue from a patient or donor; (b) enzymatically digesting the ATZ tissue with an enzyme to prepare a cell suspension; (c) optionally combining at least a portion of the cell suspension with a cryopreservation media and cryopreserving the cell suspension; and (d) combining the cell suspension of step (b) or optional step (c) with a pharmaceutically acceptable carrier.

In one embodiment, the disclosure provides a cellular composition comprising adult allogeneic or autologous ATZ cells in a pharmaceutically acceptable carrier. The ATZ cells can be prepared by a method that comprises: (a) collecting ATZ tissue from an adult human or porcine subject; (b) preparing a cell suspension in vitro by enzymatic digestion of ATZ tissue; (c) sedimenting/pelleting and then re-suspending the cells in freezing medium and cryopreserving ATZ cells in, for example, liquid nitrogen. Prior to use, the cryopreserved ATZ cells are combined with a pharmaceutically acceptable carrier, and the cell/carrier preparation is then used to treat a damaged or dysfunctional mesodermal tissue or organ. The carrier can be used to provide support for ATZ cells in a mesodermal tissue or organ.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remain operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The following examples are merely illustrative and are not intended to limit the scope or content of the disclosure in any way.

### Example 1. Isolation of Intestinal Crypts from Anorectal Tissue

This example describes protocols for isolating intestinal crypts from anorectal tissue from a pig and a human subject with Crohn's disease.

### I. Pig

An example of healthy adult white Landrace porcine tissue from the colon to anus (FIG. 1(a)) was collected in AIMV V medium (Thermo Fisher Scientific, Waltham, Massachusetts) containing antibiotics/antimycotics within 2 hours of termination and processed directly, or in some instances after overnight storage in at 4° C. The tissue was longitudinally opened and cleaned.

The dentate line was identified between the rectal mucosa and anal skin (FIG. 1(b)). The width of the tissue was 8-10 cm with a height of 1-2 cm and was pale in colouration. The epithelial layer of dentate tissue (anorectal transition zone) was excised from rectal mucosa and anal skin (FIG. 1(c)). Enzymatic treatment of minced anorectal transition zone tissue released intestinal crypts (FIG. 1(d-g)) and structures consistent with submucosal glands (FIG. 1(h-i)).

### II. Crohn's Disease Patient

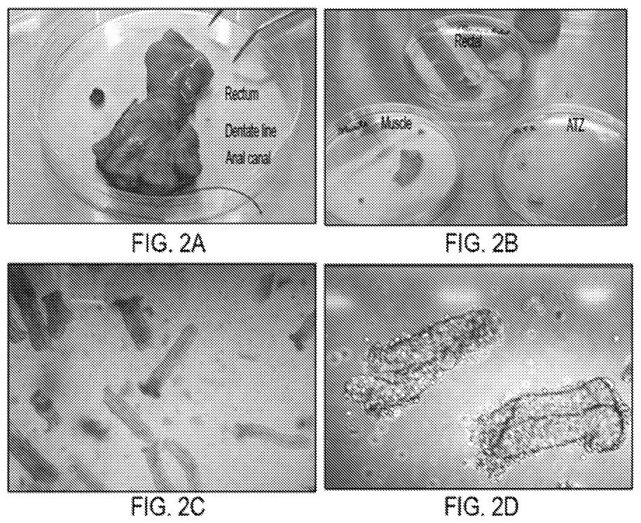

From a proctectomy tissue (FIG. 2(a)), the anorectal transition zone was excised (FIG. 2(b)), minced and enzymatically treated to release crypts (FIG. 2(c-d)).

### Example 2. Crypt Anorectal Transition Zone Organoid Development

Crypt anorectal transition zone organoid development from the pig and human tissue of EXAMPLE 1 was developed.

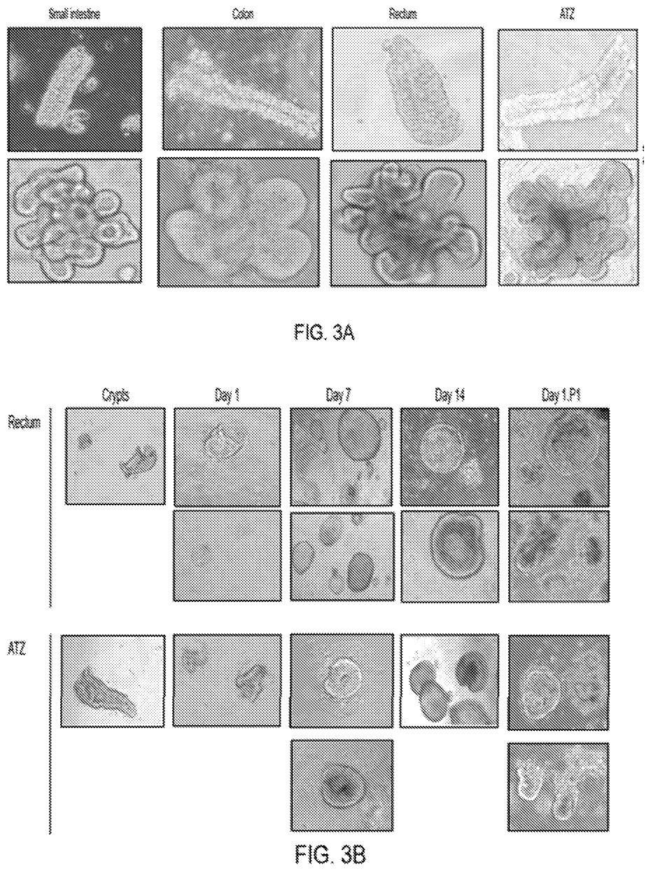

Intestinal crypts prepared in EXAMPLE 1 were embedded in Corning® Matrigel® Matrix with IntestiCult™ and developed into fully branched organoids within 1-2 weeks. The morphology of porcine crypt organoids derived from anorectal transition zone were indistinguishable from organoids derived from porcine small intestine, colon, and rectum tissue (FIG. 3(a)). Similarly, organoids derived from anorectal transition zone and rectum were indistinguishable in a Crohn's disease patient (FIG. 3(b)).

### Example 3. Increased Progenitor Capacity of Single Cell Preparations of Porcine ATZ Crypt Cells Compared to Rectal Crypt Cells

This example demonstrates that single cell preparations of porcine ATZ crypt cells have increased progenitor capacity, assessed by plating efficiency, compared to rectal crypt cells for the purpose of describing and illustrating certain examples and embodiments of the present disclosure.

The plating efficiencies (organoids formed per cells plated) of single cell preparations of porcine ATZ and rectal crypt cells were determined for an input of 500, 1,000 and 2,000 viable crypt cells per well in 24 well, flat base, suspension surface culture plates (Sarstedt, Numbrecht, Germany) and monitored weekly for 4 weeks. IntestiCult™ was refreshed once at the end of week 2 without a change in Corning® Matrigel® Matrix.

Few organoids formed from 500 input cells in either group.

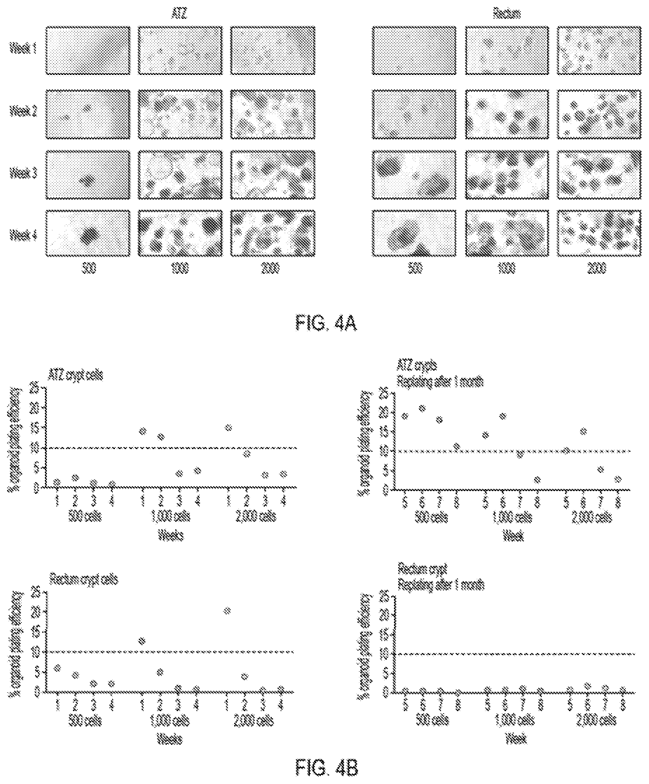

At week 1, the appearance (FIG. 4(a)) and frequency (FIG. 4(b)) of organoids forming was similar for ATZ-derived and rectal-derived organoids for cultures initiated with 1,000 or 2,000 cells. At week 2, rectal-derived organoid cultures contained more fully differentiated organoids (dark clusters of cells), whereas ATZ-derived organoid cultures contained more undifferentiated viable organoids (cystic organoid structures) (FIG. 4(a)).

Few viable organoids were detected at weeks 3 and 4 in cultures initiated with 1,000 and 2,000 cells: 3-4% for ATZ and 0-1% for rectum (FIG. 4(b)).

After 4 weeks, cultures were harvested and reinitiated with 500, 1,000, or 2,000 viable cells to determine plating efficiencies for an additional 4 weeks. IntestiCult™ was refreshed at week 6 without a change in Corning® Matrigel® Matrix. Plating efficiencies were assessed at weeks 5, 6, and 7 (FIG. 4(b)). The highest plating efficiency was observed with 500 replated ATZ-derived cells over weeks 5-7 (18%-21%). Plating efficiencies for 1,000 and 2,000 replated ATZ-derived cells peaked at week 6 (19% and 15%, respectively) and decreased at week 7 (9% and 5%, respectively).

Plating efficiencies for all replated rectal-derived cells was <2% over weeks 5-7. Taken together, these studies show increased progenitor capacity, determined by plating efficiencies, in porcine ATZ crypt cells compared to rectum crypt cells. These results are consistent with the existence of a long-term repopulating ATZ stem cell population.

### Example 4. Protein Expression of Stem Cell Markers of Embryonic Developmental Lineages on Freshly Isolated ATZ Crypts and ATZ-Derived Crypt Organoids

This example shows protein expression detected by immunocytochemistry of stem cell markers of embryonic developmental lineages on freshly isolated porcine crypts and crypt organoids for the purpose of describing and illustrating certain examples and embodiments of the present disclosure.

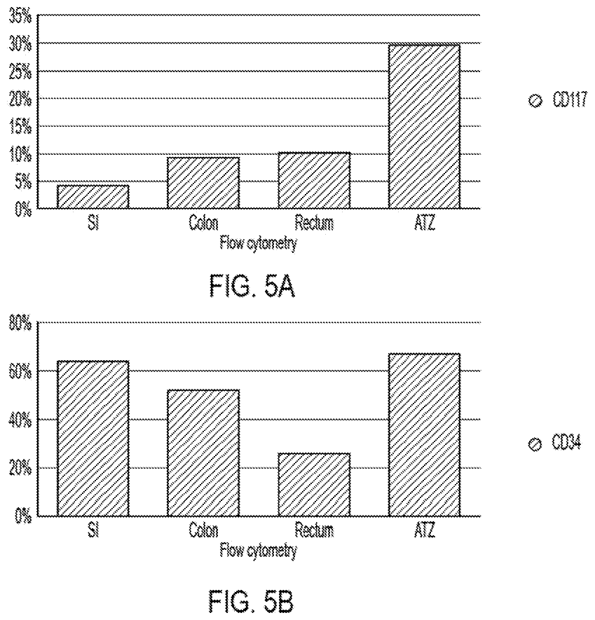

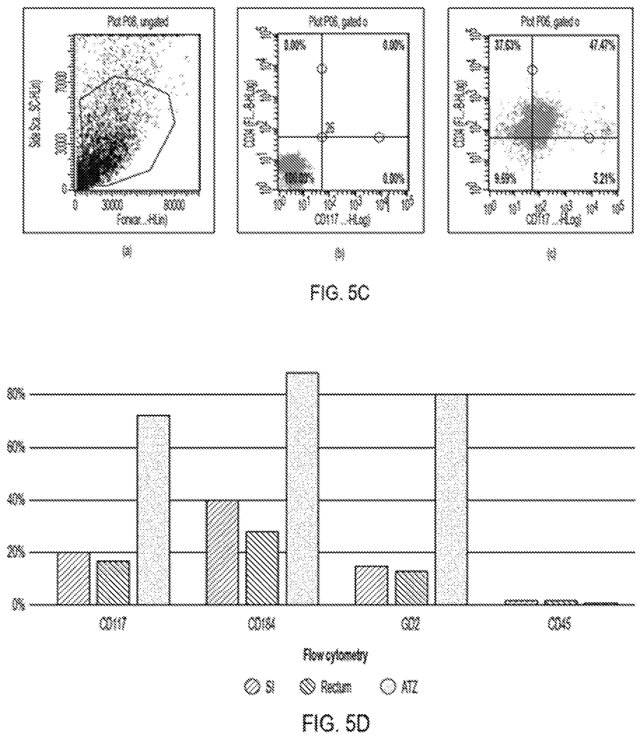

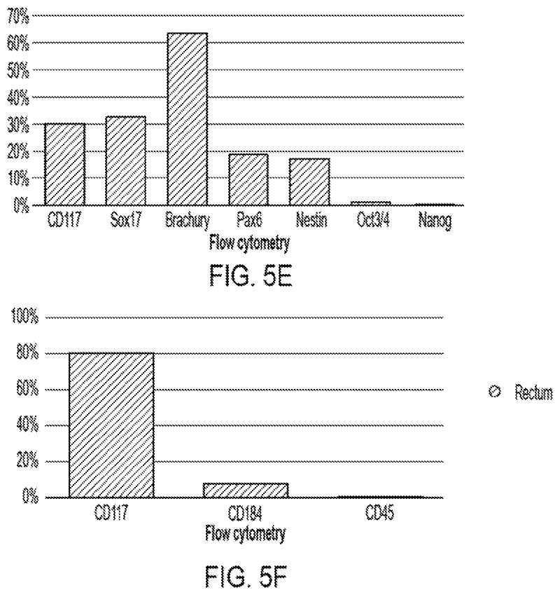

Freshly isolated porcine ATZ crypts. Cell surface stem cell markers were examined by flow cytometry on single cell preparations from freshly isolated porcine crypts derived from the small intestine, colon, rectum, and ATZ tissue. Expression of the stem cell and progenitor cell growth factor receptor, KIT (CD117), was expressed at 3-5-fold higher levels in ATZ crypt cells compared to small intestine, colon, and rectum crypt cells (FIG. 5(a)). Crypt cells from these tissues all expressed the stem cell and progenitor cell marker, CD34, to varying extent, with ATZ crypt cells expressing the highest levels (FIG. 5(b)).

In a double labelling experiment, 85% of ATZ crypt cells expressed CD34, 53% of cells expressed CD117; 47% of cells co-expressed CD34 and CD117; and of the CD117 expressing cells, 88% co-expressed CD34 (FIG. 5(c)).

Porcine ATZ crypt organoids. Cell surface stem cell markers were examined by flow cytometry on single cell preparations of fully differentiated porcine crypt organoids derived from the small intestine, rectum, ATZ cells after 10 days of culture. Expression of CD117, CD184 and GD2 were increased by more than 3-fold in ATZ-derived organoids compared to SI- and rectal-derived organoids. The haematopoietic marker, CD45, was undetected in all crypt organoids (FIG. 5(d)).

Because KIT and CXCR4 are markers of definitive endoderm and GD2 is a marker of primitive mesoderm, further investigation of the developmental lineage origins of freshly isolated ATZ crypt cells was undertaken. Intracellular protein markers confirmed the expression of transcriptional factors associated with the endoderm (SOX17), mesoderm (TBXT), and ectoderm (PAX6 and NESTIN) lineages. However, expression of transcriptional factors associated with pluripotency (OCT4 and NANOG) were not detected by flow cytometry (FIG. 5(e)).

Taken, together these results indicate porcine ATZ crypt cell populations express stem cell and progenitor protein markers for all 3 developmental lineages, and that markers for endoderm and mesoderm were maintained in ATZ crypt organoids.

### Example 5. mRNA Expression Profiling of Porcine ATZ Crypt Cells

This example shows mRNA expression profiling of porcine ATZ crypt cells for the purpose of describing and illustrating certain examples and embodiments of the present disclosure.

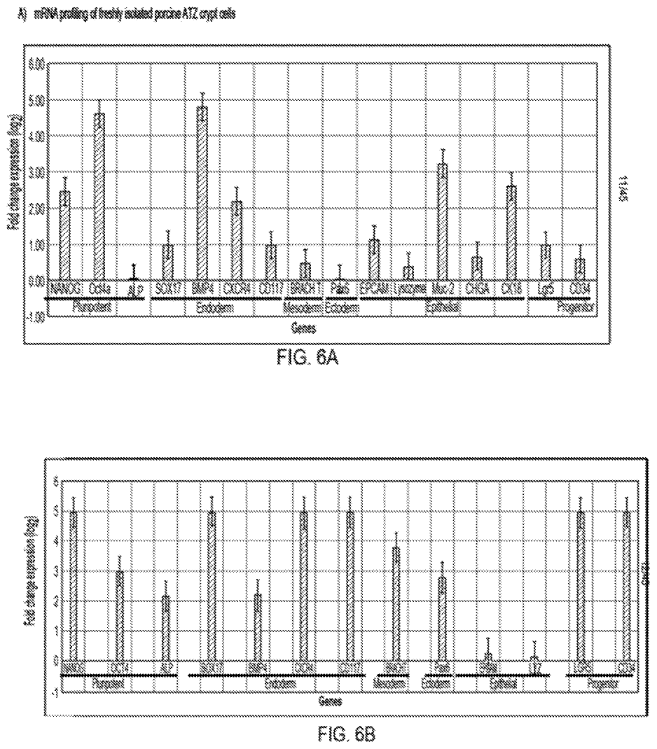

Fresh porcine ATZ crypt cells. Transcriptional profiling of fresh porcine ATZ crypt cells confirmed basal expression of markers for mature epithelial cells (EPCAM, LYZ, MUC2, CHGA, CK18); stem cells and progenitor cells (CD34, LGR5); and developmental stem cells of the endoderm (SOX17), mesoderm (TBXT), and ectoderm (PAX6) lineages; and pluripotent stem cells (NANOG, OCT4A) (FIG. 6(a)). Although mRNA expression of alkaline phosphatase (ALP) was not detected, the enzyme was detected by immunocytochemistry in

Porcine ATZ-derived crypt organoids. Transcriptional profiling of ATZ-derived crypt cells cultured in IntestiCult™ retained mRNA expression for markers of stem cells of all three developmental lineages (FIG. 6(b)). Lower levels of mRNA expression for markers of mature epithelial cells were detected when compared to fresh ATZ cells. mRNA expression levels were normalised to glyceraldehyde 3-phosphate dehydrogenase (GAPDH).

Taken together, transcriptional profiling of fresh ATZ crypts and organoids confirmed the presence of markers for stem cells of all three developmental lineages.

### Example 6. Culture Medium for Maintaining and Expanding Multipotent Porcine ATZ Stem Cells

This example demonstrates in vitro culture conditions for maintaining and expanding multipotent ATZ cells for the purpose of describing and illustrating certain examples and embodiments of the present disclosure.

Freshly isolated porcine ATZ crypt single cells were cultured for 14 days in either (1) a feeder-free medium that maintains human embryonic stem cells (ESCs) and induced pluripotent stem cells (iPSCs) in an undifferentiated state (mTeSR™) or in (2) human intestinal IntestiCult™.

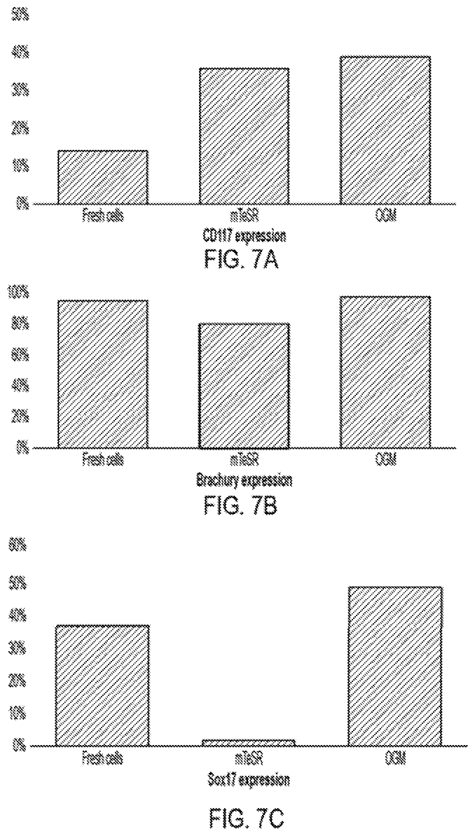

The protein expression levels of multipotential stem cell markers described in EXAMPLE 4 were assessed by flow cytometry before culture (day 0) and after 14 days in culture. The stem cell and progenitor marker CD117 was expressed on 14% ATZ crypt cells before culture (day 0) and increased to 35% in mTeSR™ and 28% human IntestiCult™ at day 14 (FIG. 7(a)).

The intracellular marker for mesoderm (Brachyury) was expressed on 97% of ATZ crypt cells before culture (day 0) and the expression levels were maintained at 99% in mTeSR™ and 81% in human IntestiCult™ at day 14 (FIG. 7(b)).

The intracellular marker for endoderm, SOX17, was expressed on 34% of ATZ crypt cells before culture (day 0) and <1% expression in mTeSR™ medium and 49% in human IntestiCult™ at day 14 (FIG. 7(c)).

These results indicate that IntestiCult™ may be preferable for maintaining ATZ crypt cells expressing markers of both endoderm and mesoderm developmental lineages.

### Example 7. In Vitro Differentiation of Porcine ATZ Cell-Derived Crypts into all Three Developmental Lineages

This example demonstrates that porcine ATZ-derived crypts differentiate in vitro into all three developmental lineages (endoderm, mesoderm, and ectoderm) for the purpose of describing and illustrating certain examples and embodiments of the present disclosure.

Expression of protein and mRNA markers in all three developmental lineages of fresh porcine ATZ crypt stem cells in EXAMPLE 4 and EXAMPLE 5, respectively, led to studies to determine if freshly isolated ATZ crypt cells had the capacity to generate mature cells of the endoderm lineage (gut mucosa), mesoderm (blood vessels), and ectoderm (keratinocytes).

### Example 7.1. Endoderm Differentiation Potential of Fresh Porcine ATZ Crypts

Protein expression levels of mature endoderm cell markers of gut mucosa were assessed after two weeks of culturing single cell preparations of fresh ATZ crypts embedded in Corning® Matrigel® Matrix with IntestiCult™ to generate organoids.

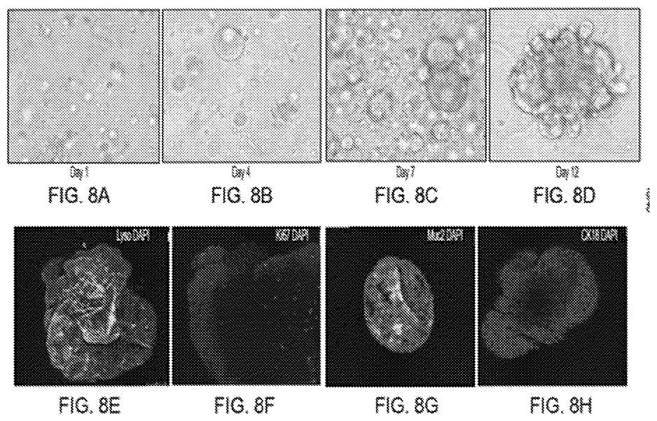

Immunostaining of intact organoids was visualised for expression of lysozyme, proliferating cells (K167), secretory cells (MUC2), and cytokeratin 18 (CK18) which is expressed in the single layer of gut epithelium (FIG. 8(a-h)).

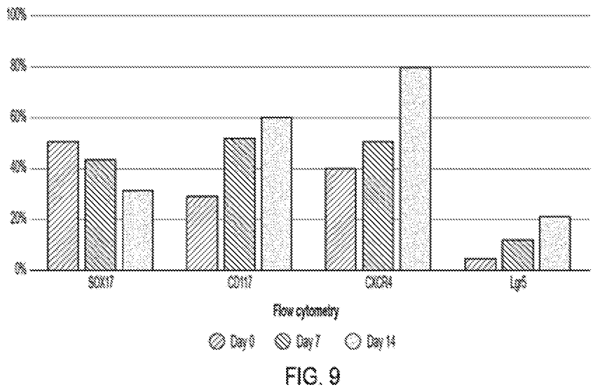

Flow cytometric analysis of fresh ATZ crypt cells showed that SOX17 decreased from 52% at day 0 to 34% at day 14; and that KIT (CD117) and CXCR4 (CD184) increased by twofold during culture (FIG. 9). The intestinal stem cell marker, LGR5, was expressed at 3% on day 0 and increased to 22% at day 14.

Taken together these results support that fresh ATZ crypts have the capacity to generate mature cell types derived from the endodermal lineage.

### Example 7.2. Mesoderm Differentiation Potential of Porcine ATZ Crypts

Protein expression levels of mature mesoderm cell markers were assessed after two weeks of culturing single cell preparations of fresh ATZ crypts in MethoCult™ to test their capacity to generate progeny in the haematopoietic lineage. No haematopoietic colonies formed in the non-adherent, methylcellulose layer.

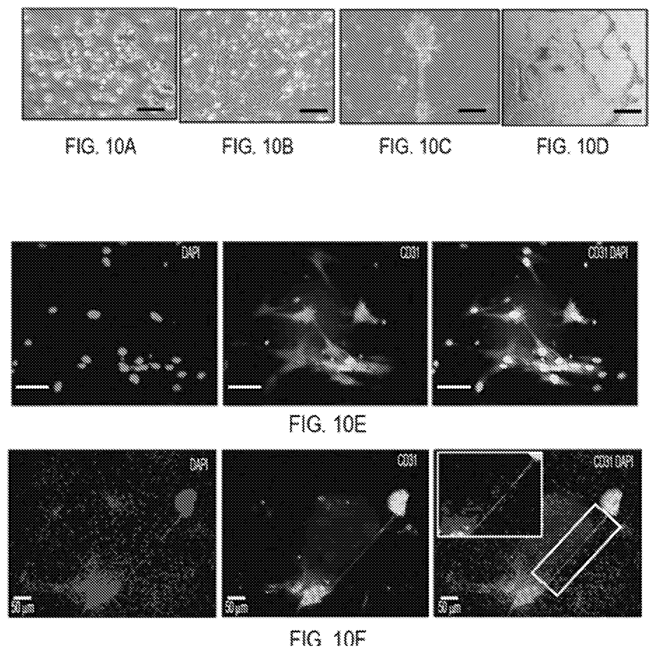

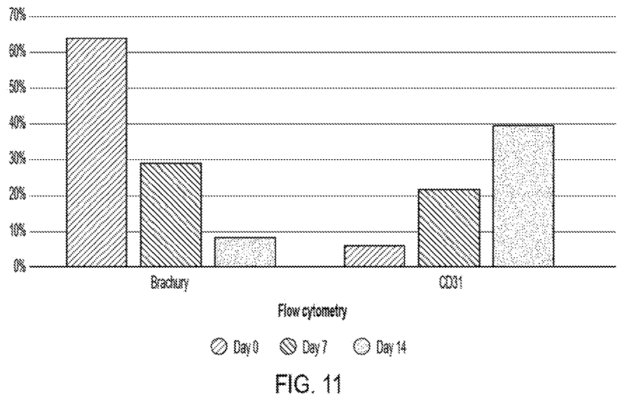

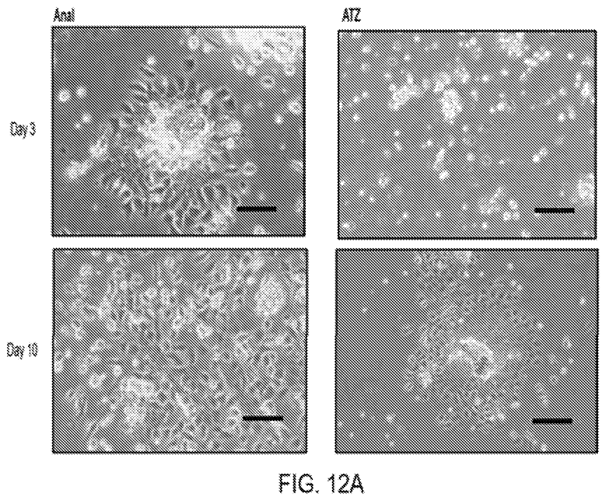

However, single ATZ crypt-derived cells (FIG. 10(a)) formed clusters in the adherent layer at day 3 (FIG. 10(b)) and initiated and formed a network of blood vessel-like tubular structures at day 10 (FIG. 10(c-d)). Immunostaining with the endothelial intracellular marker, CD31 (PECAM), was detected at the initiation and formation of a network of blood vessel-like tubular structures (FIG. 10(e-f)). Flow cytometric analysis of fresh ATZ cells showed that brachyury expression decreased from 67% at day 0 to 23% at day 7 and decreasing further to 7% on day 14; CD31 expression increased from 5% at day 0 to 19%, and further at day 7 to 37% at day 14 (FIG. 11).

Taken together these results support that fresh ATZ crypts have the capacity to generate mature cell types derived from the mesoderm lineage.

### Example 7.3. Ectoderm Differentiation Potential of Porcine ATZ Crypts

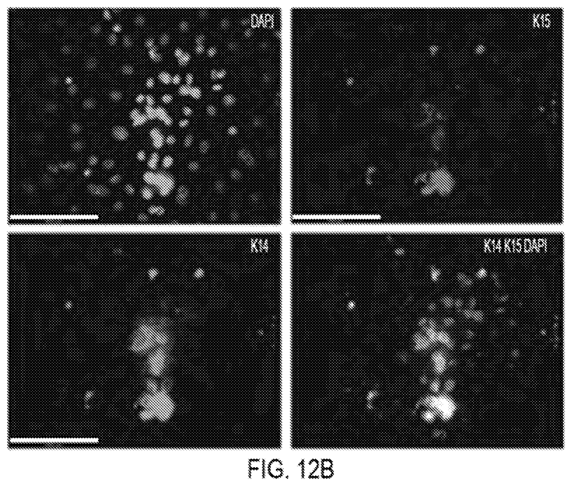

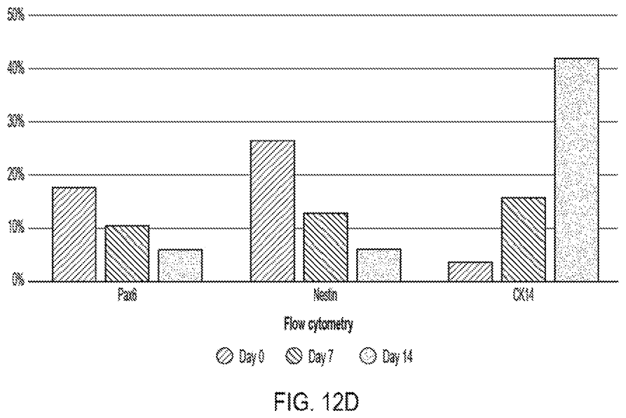

Protein expression levels of mature ectodermal cell markers were assessed after two weeks culturing single cell preparations of fresh ATZ crypts and anal skin in keratinocyte differentiation medium (KSFM, Sigma-Aldrich, St, Louis, Missouri). Cell clusters developed at day 3 and a cobblestone-like adherent layer with keratinocyte morphology formed in anal skin and ATZ cell cultures by day 10 (FIG. 12(a)). Co-expression of the keratinocyte markers, CK14 and CK15, were detected by immunostaining of anal skin adherent cultured cells (FIG. 12(b)) and ATZ adherent cultured cells (FIG. 12(c)).

Flow cytometric analysis of ATZ adherent culture cells showed that the ectodermal lineage marker, PAX6, expression decreased in culture from 18% at day 0 to 6% at day 7 and 5% at day 14 (FIG. 12(d)). CK14 expression on ATZ adherent culture cells increased over time in culture from 3% on day 0 to 18% on day 7 and 42% on day 14 (FIG. 12(d)).

Taken together these results support that fresh ATZ crypts have the capacity to generate mature cell types derived from the ectoderm lineage.

### Example 8. Absence of Multipotential Stem Cells of all Three Developmental Lineages in Porcine Small Intestine Crypts

This example demonstrates that porcine small intestine crypts have the potential to generate mature cells of the endodermal lineage, as expected but not mesodermal, and ectodermal lineages in vitro using the same methods as described in EXAMPLE 7 for porcine ATZ crypts for the purpose of describing and illustrating certain examples and embodiments of the present disclosure.

Small intestine single crypt cells were cultured for two weeks to promote differentiation of endoderm (IntestiCult™), mesoderm (MethoCult™) and ectoderm (KSFM, Sigma-Aldrich).

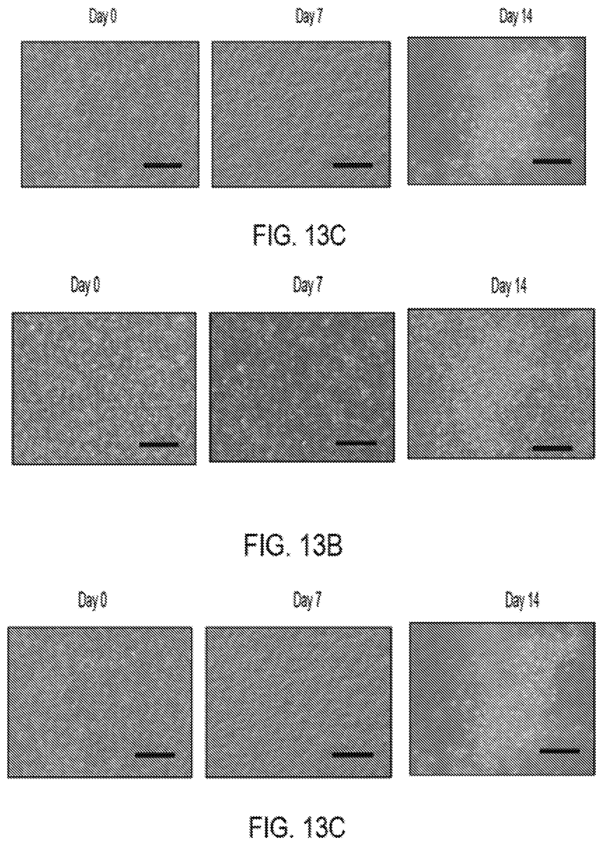

Small intestine crypt cells generated organoids (IntestiCult™), as expected (FIG. 13(a)). However, no growth was detected for small intestine crypt cells at days 7 or 14 when cultured in MethoCult™ (FIG. 13(b)) or KFSM (FIG. 13(c)).

Taken together, these results are consistent with the absence of multipotential stem cells in porcine small intestine crypts capable of generating cell types of all three developmental lineages, using the assays demonstrated for ATZ crypt cells.

### Example 9. In Vitro Embryoid Body Assay to Assess the Pluripotency of Porcine ATZ Crypt Cells

This example demonstrates that single cell preparations of fresh ATZ crypts cultured in feeder-free mTeSRT medium can promote embryoid body formation for the purpose of describing and illustrating certain examples and embodiments of the present disclosure.

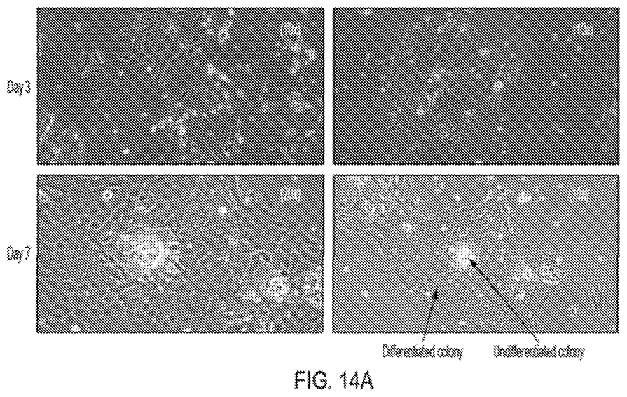

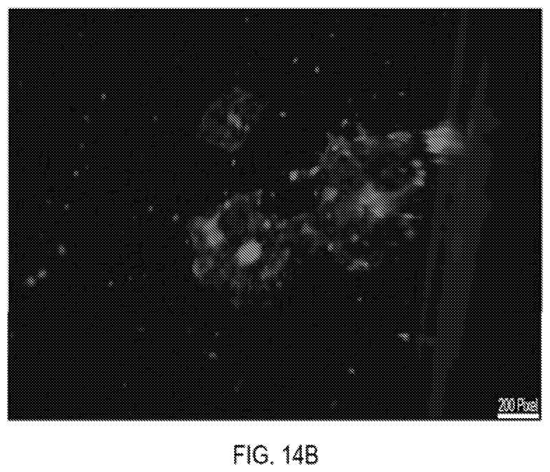

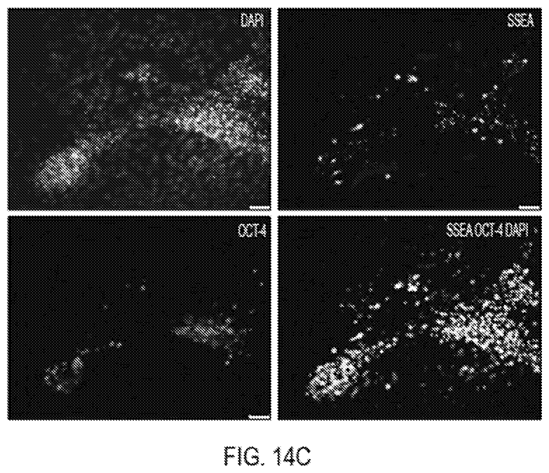

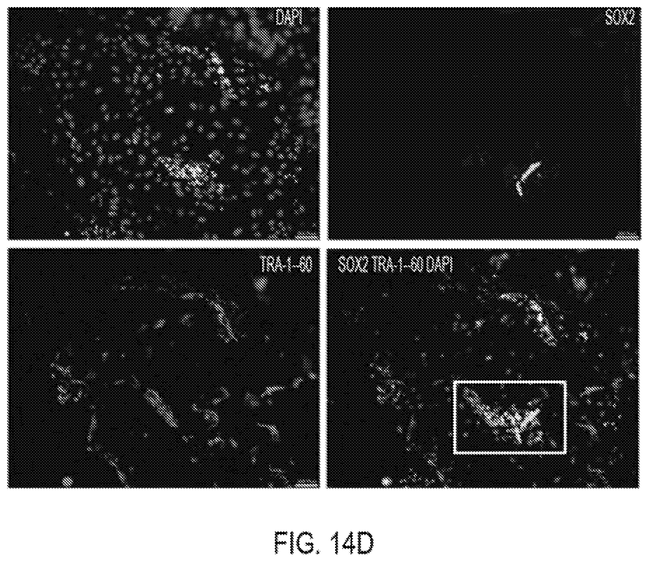

Fresh ATZ crypt single cells plated at a high density generated a cobblestone-like adherent layer by day 3; colonies of undifferentiated adherent cells developed by day 7; and differentiated adherent cells were generated and migrated away from the undifferentiated cell colonies (FIG. 14(a)).

Visualisation of alkaline phosphatase staining confirmed stem cell-like properties consistent with embryoid bodies (FIG. 14(b)). Furthermore, pluripotent stem cell markers, OCT4 and SSEA4 (FIG. 14(c)) and SOX2 and TRA-1-60 (FIG. 14(d)) were detected by immunocytochemistry at day 5.

Taken together, the results of the in vitro embryoid body assay are consistent with ATZ crypt cells exhibiting pluripotent stem cell-like properties.

### Example 10. Isolation of Stem Cells from Porcine Kidney Tissue

This example describes protocols for isolating stem cells from kidney tissue.

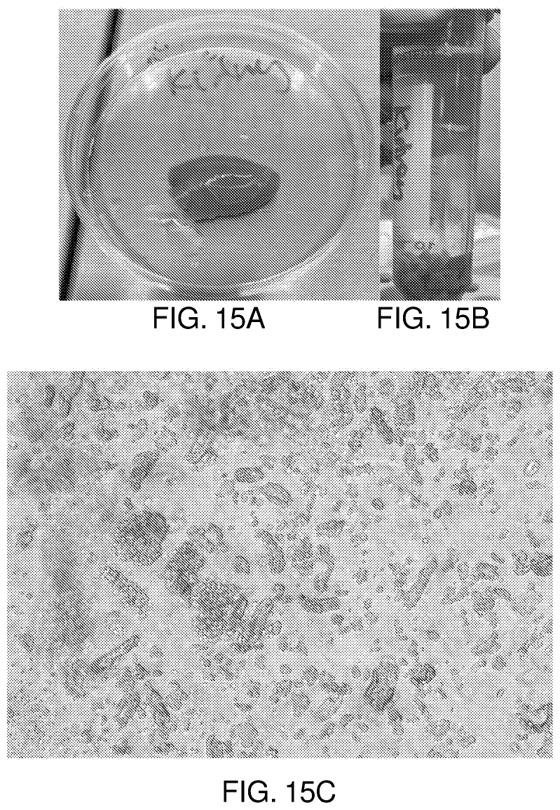

An example of healthy adult white Landcross porcine tissue from the kidney was collected in AIMV medium (Thermo Fisher Scientific) containing antibiotics/antimycotics (AA) within 2 hours of termination and processed after overnight storage at 4° C. (FIG. 15(a)). The tissue was diced into 1 cm pieces and transferred to a 50 ml conical tube containing PBS+AA+Nystatin solution (FIG. 15(b)). The tissue was washed repeatedly until the PBS solution was clear of particulates. Enzymatic treatment of kidney tissue to release single cells that include stem cells and progenitors (FIG. 15(c))

### Example 11. Establishment and Maintenance of Organoids Derived from Porcine ATZ Crypts and Kidney Tissues Cultured in StemDiff Kidney™ Organoid Kit

This example describes how porcine ATZ crypts isolated in EXAMPLE 1 and kidney stem cells in EXAMPLE 10 were cultured in StemDiff Kidney™ Organoid Kit to promote kidney organoid development and maintenance for the purpose of describing and illustrating certain examples and embodiments of the present disclosure.

ATZ crypts and kidney stem cells were separately embedded in Corning® Matrigel® Matrix with StemDiff Kidney™ Organoid Kit.

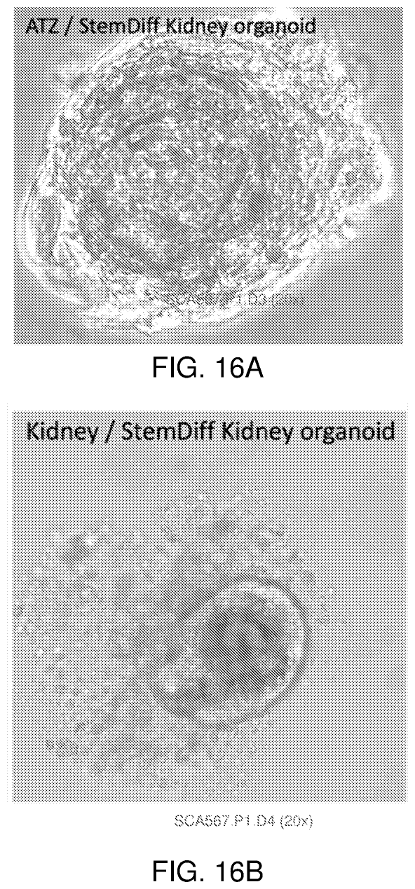

Similar compact organoid structure morphology was observed for ATZ crypts ((FIG. 16(a)) and kidney stem cells ((FIG. 16(b)) cultured in StemDiff Kidney™ Organoid Kit.

Taken together these experimental results indicate that porcine ATZ crypt cells differentiated into kidney cells in vitro.

### Example 12. mRNA Expression Profiling Markers Expressed During Development of Porcine ATZ Crypt-Derived Kidney-Like Organoids

This example shows mRNA expression of markers detected by gradient PCR during development of kidney-like organoids generated from porcine ATZ crypts EXAMPLE 11 for the purpose of describing and illustrating certain examples and embodiments of the present disclosure.

Transcriptional profiling of organoids derived from porcine ATZ crypt cells cultured in StemDiff Kidney™ Organoid Kit genes for the master regulators of kidney stem cell and progenitor cell development and mature cell function (TABLE 1).

TABLE 1

| Gene | Cells | Description |
|---|---|---|
| EYA1 | Stem/progenitors | Eyes absent homolog 1 is protein involved |
| | | in kidney development |
| HAND1 | Stem/progenitors | Heart- and neural crest derivatives- |
| | | expressed protein 1 is a transcription |
| | | protein involved in kidney development |
| LHX1 | Stem/progenitors | LIM-class homeobox transcription factor 1 |
| NPHS1 | Stem/progenitors | Nephrin is necessary for the proper |
| | | functioning of the renal filtration barrier |
| NPHS2 | Stem/progenitors | Podocin interacts with NPHS1 |
| PAX2 | Stem/progenitors | Paired box gene 2 involved in developing |
| | | kidney |
| SALL1 | Stem/progenitors | SALL1 is zinc finger protein repressor is |
| | | essential kidney development |
| SIX2 | Stem/progenitors | Six2-expressing cells give rise to all cell- |
| | | types of the main body of the nephron, |
| | | during all stages of nephrogenesis |

### Example 13. Establishment and Maintenance of Organoids Derived from Porcine ATZ Crypts Cultured in Human STEMdiff™ Ventricular Cardiomyocyte Differentiation Kit

This example describes how porcine ATZ crypts isolated in EXAMPLE 1 were cultured in STEMdiff™ Ventricular Cardiomyocyte Differentiation Kit in promote ventricular cardiomyocyte organoid development and maintenance for the purpose of describing and illustrating certain examples and embodiments of the present disclosure.

ATZ crypts were embedded in Corning® Matrigel® Matrix with different components in the STEMdiff™ Ventricular Cardiomyocyte Differentiation Kit.

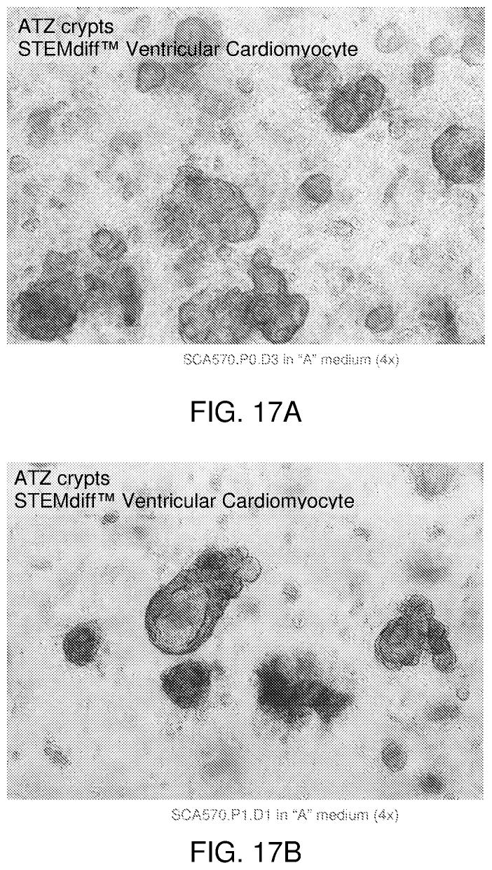

ATZ-derived organoids developed in each component in the STEMdiff™ Ventricular Cardiomyocyte Differentiation Supplement A. FIG. 17(a) shows cardiomyocyte organoids developing after 3 days in cultures, FIG. 17(b) shows cardiomyocyte organoids forming 1 day after passage.

Taken together these experimental results indicate that porcine ATZ crypt cells differentiated into cardiomyocytes cells in vitro.

### Example 14. MRNA Expression Profiling Markers Expressed During Development of Porcine ATZ Crypt Derived STEMdiff™ Ventricular Cardiomyocyte Differentiation Kit

This example shows mRNA expression of markers detected by gradient PCR during development of kidney organoids generated from porcine ATZ crypts and kidney tissue in EXAMPLE 11 for the purpose of describing and illustrating certain examples and embodiments of the present disclosure.

Transcriptional profiling of organoids derived from porcine ATZ crypt cells cultured in StemDiff Kidney™ Organoid Kit genes for the master regulators of kidney stem cell and progenitor cell development and mature cell function (TABLE 2).

TABLE 2

| Gene | Cells | Description |
|---|---|---|
| KLF4 | Cardiomyocytes | Kruppel-like factor 4 is critical for |
| | | transcriptional control of cardiac |
| | | mitochondrial homeostasis |
| ISL1 | Cardiovascular | ISL1 cardiovascular progenitor cells for |
| | progenitors | cardiac repair after myocardial infarction |
| MEF2C | Stem/progenitors | MEF2C is an essential transcriptional |
| | | regulator of cardiovascular |
| | | development |
| NKX2.5 | Stem/progenitors | Nkx2.5 activity is essential for |
| | | cardiomyogenesis |
| PDGFRA | Fibroblasts | PDGFRα signalling in cardiac |
| | | fibroblasts modulates quiescence, |
| | | metabolism, and self-renewal, and |
| | | promotes anatomical and functional |
| | | repair |
| PECAM | Cardiac | PECAM mediates endothelial- |
| | endothelium | cardiomyocyte communication and |
| | | regulates cardiac function |

### Example 15. Establishment and Maintenance of Differentiated Multinucleated Myotubes Derived from Porcine ATZ Crypts

This example describes how porcine ATZ crypts isolated in EXAMPLE 1 were cultured in MyoCult™ to promote myoblasts, myocytes, and myotube development and maintenance for the purpose of describing and illustrating certain examples and embodiments of the present disclosure.

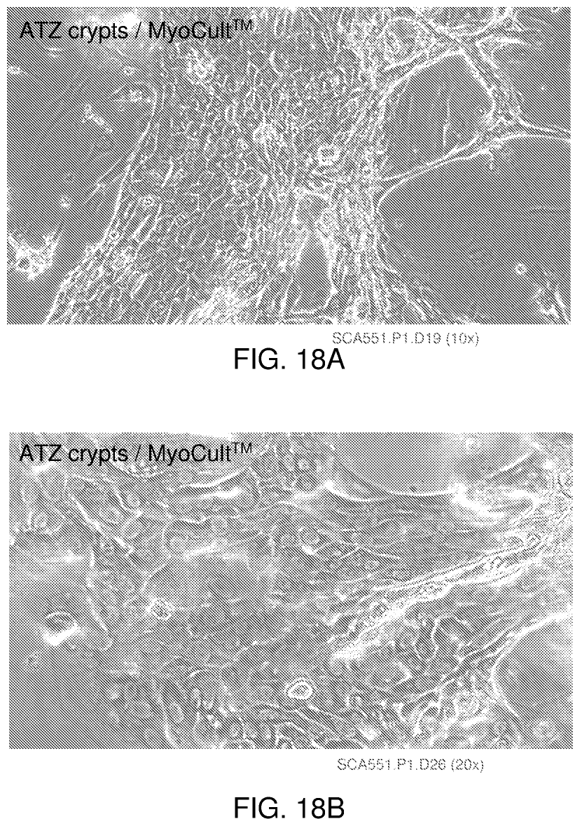

ATZ crypts cultured in MyoCult™ expansion and differentiation medium generated a monolayer with a morphology under brightfield microscopy differentiated multinucleated myotubes after passage at day 19 (FIG. 18(a) and day 26 (FIG. 18(b)).

Taken together these experimental results indicate that porcine ATZ crypt cells differentiated into multinucleated myotubes in vitro.

### Example 16. Establishment and Maintenance of Chondrocytes Derived from Porcine ATZ Crypts Cultured in MesenCult™ ACF Chondrogenic Differentiation Kit

This example describes how porcine ATZ crypts isolated in EXAMPLE 1 were cultured in MesenCult™ ACF Chondrogenic Differentiation Kit to promote chondrocyte development and maintenance for the purpose of describing and illustrating certain examples and embodiments of the present disclosure.

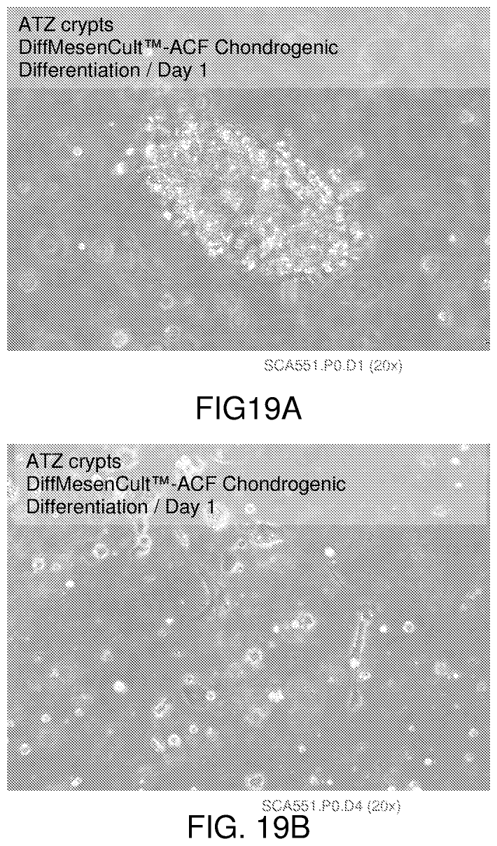

Clusters of cells were observed when ATZ crypts were cultured in MesenCult™ ACF Chondrogenic Differentiation Kit for 1 day (FIG. 19(a)) and 4 days (FIG. 19(b)).

Taken together these experimental results indicate that porcine ATZ crypt cells differentiated into chondrocytes in vitro.

### Example 17. Isolation of Intestinal Crypts from Anorectal Tissue of an Idiopathic Perianal Fistula Patient

This example describes a protocol for isolating intestinal crypts from anorectal tissue of a human subject with an idiopathic perianal fistula.

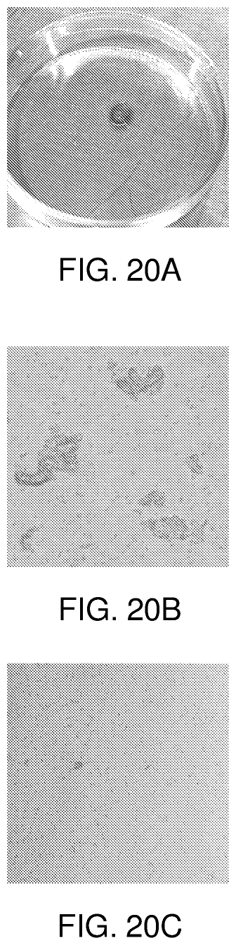

A biopsy from anorectal tissue of an idiopathic perianal fistula patient was collected in AIMV medium containing antibiotics/antimycotics within 2 hours of termination and processed directly, or in some instances after overnight storage in at 4° C. (FIG. 20(a)). Enzymatic treatment of minced ATZ tissue released intestinal crypts (FIG. 20(b)) and single cell preparations were prepared (FIG. 20(c)).

### Example 18. mRNA Expression Profiling by qPCR of Freshly Isolated ATZ Crypt from an Idiopathic Perianal Patient

This example shows mRNA expression profiling by qPCR of idiopathic perianal patient ATZ crypt for the purpose of describing and illustrating certain examples and embodiments of the present disclosure.

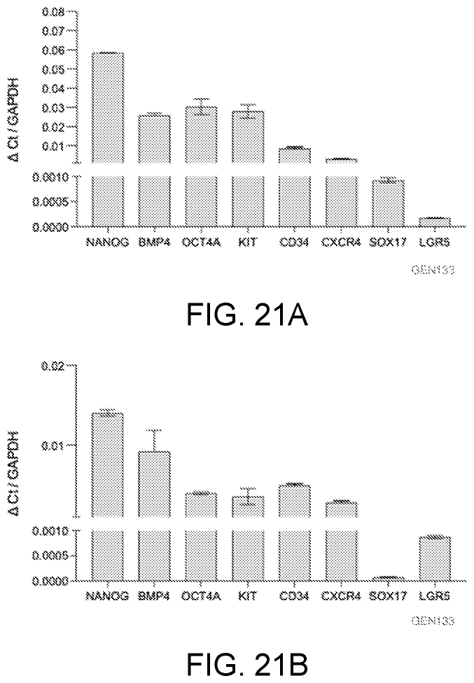

Transcriptional profiling of freshly isolated ATZ crypts from two idiopathic perianal patients show that ATZ crypt cells expressed markers for pluripotent stem cells (NANOG, OCT4A), multipotent stem cells and progenitor cells (BMP4, KIT, CD34, CXCR4, LGR5), and developmental stem cells of the endoderm (SOX17) (FIG. 21(a-b)).

Taken together with mRNA profiling of porcine ATZ crypts in EXAMPLE 5, these results confirm that ATZ crypts contain populations of pluripotent and multipotent stem cells.

### Example 19. Crypt Anorectal Transition Zone Organoid Development from an Idiopathic Perianal Fistula Patient

Crypt ATZ organoid development from human tissue of EXAMPLE 17 was developed.

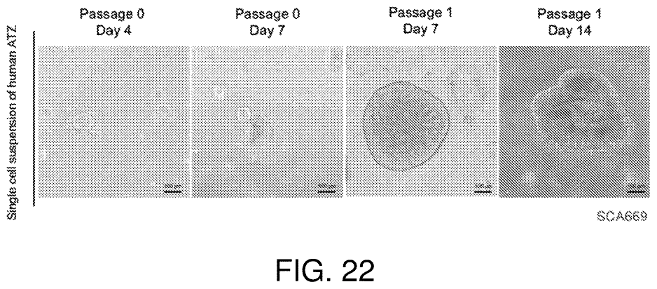

Crypts or single cell preparations of ATZ tissue prepared in EXAMPLE 17 were embedded in Corning® Matrigel® Matrix with IntestiCult™ and developed into fully formed organoids in 1-3 weeks (FIG. 22). Clusters of cells formed on day 4 and organized structures formed on day 7 (both at passage 0); compact, thickened organoids formed on day 7 and 14 of passage 1.

### Example 20. Establishment and Maintenance of Organoids Derived from Idiopathic Perianal Fistula Patient ATZ Cells Cultured in STEMdiff™ Kidney Organoid Kit

This example describes how organoids derived idiopathic perianal fistula patient ATZ crypts or single cell preparations in EXAMPLE 17 were cultured in STEMdiff™ Kidney Organoid Kit (STEMCELL Technologies) to promote kidney organoid development and maintenance for the purpose of describing and illustrating certain examples and embodiments of the present disclosure.

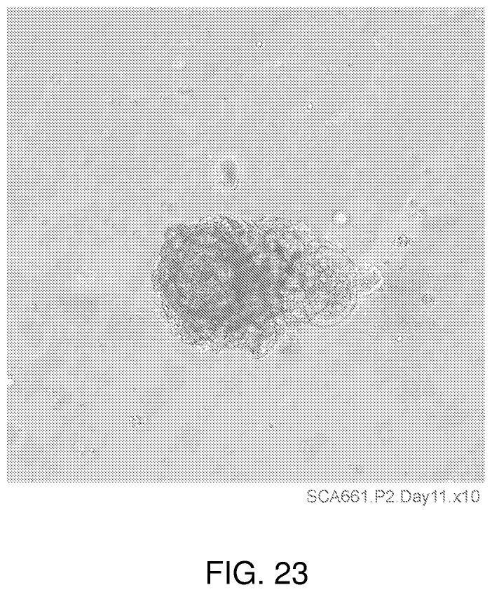

ATZ crypts and single cells were embedded in Corning® Matrigel® Matrix and cultured with STEMdiff™ Kidney Organoid Kit. Organised, complex organoids formed after 2 passages (FIG. 23).

Small ring structures formed from ATZ-derived crypts at day 4, filled in ring structures at day 7, and developed organoids at day 14 (FIG. 19(a)). Small cystic organoid structures formed from liver-derived crypts at day 3, larger cystic organoid structures formed at days 1 and 4 after passage (FIG. 19(b)). Similar cystic organoid structures with a one-sided budding outgrowth were observed for ATZ crypts and liver stem cells grown in HepatiCult™ (FIG. 19(c)).

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A cellular composition for use in cell transplantation to treat a damaged or dysfunctional mesodermal tissue in a subject, comprising:
a mixed population of ATZ cells comprising multipotent ATZ stem cells and mesodermal progenitor ATZ cells in a pharmaceutically-acceptable carrier or exogenous biocompatible scaffold.

2. The cellular composition of claim 1, wherein:
the multipotent ATZ stem cells comprise at least 5% of the total ATZ cells.

3. The cellular composition of claim 1 or claim 2, wherein:
the multipotent ATZ stem cells do not express significant levels of CD45.

4. A method of producing a cellular composition for use in cell transplantation to treat a damaged or dysfunctional mesodermal tissue in a subject, comprising:
(a) obtaining a sample of ATZ cells that are autologous or allogeneic to the subject;
(b) isolating a mixed population of ATZ cells comprising multipotent ATZ stem cells and mesodermal progenitor ATZ cells from the sample; and
(c) expanding the multipotent ATZ stem cells in a minimal growth medium (MGM).

5. The method of claim 4, wherein:
the multipotent ATZ stem cells do not express significant levels of CD45.

6. The method of any one of claims 4-5, wherein:
the multipotent ATZ stem cells express markers CD34, CD117 and CD184

7. The method of any one of claims 4-6, wherein:
wherein the step of expanding the multipotent ATZ stem cells comprises replating the ATZ cells through at least 5 passages in MGM.

8. The method of any one of claims 4-7, further comprising:
(d) culturing the ATZ cells in an mesodermal lineage-specific medium (LSM) to promote at least partial differentiation of the multipotent ATZ stem cells to produce an expanded population of mesodermal progenitor ATZ cells.

9. The method of claim 8, wherein:
the damaged or dysfunctional mesodermal tissue is cardiac muscle tissue and the LSM comprises a cardiomyocyte-specific LSM.

10. The method of claim 9, wherein:
wherein the LSM promotes differentiation of multipotent ATZ stem cells to produce a population of ATZ stem cell-derived cardiac muscle organoid cells.

11. The method of claim 8, wherein:
the damaged or dysfunctional mesodermal tissue is skeletal muscle and the LSM comprises a skeletal muscle-specific LSM.

12. The method of claim 11, wherein:
wherein the LSM promotes differentiation of multipotent ATZ stem cells to produce a population of ATZ stem cell-derived skeletal muscle organoid cells.

13. The method of claim 8, wherein:
the damaged or dysfunctional mesodermal tissue is cartilage tissue and the LSM comprises an chondrocyte-specific LSM.

14. The method of claim 13, wherein:
the LSM promotes differentiation of multipotent ATZ stem cells to produce a population of ATZ stem cell-derived chondrocyte organoid cells.

15. The method of claim 8, wherein:
the damaged or dysfunctional mesodermal tissue is kidney tissue and the LSM comprises a kidney-specific LSM.

16. The method of claim 15, wherein:
wherein the LSM promotes differentiation of multipotent ATZ stem cells to produce a population of ATZ stem cell-derived kidney organoid cells.

17. A method treating damaged or dysfunctional mesodermal tissue in a subject, comprising:
(a) obtaining a sample of ATZ cells that are autologous or allogeneic to the subject;
(b) isolating a mixed population of ATZ cells comprising multipotent ATZ stem cells and mesodermal progenitor ATZ cells from the sample;
(c) expanding the multipotent ATZ stem cells within the mixed population in a minimal growth medium (MGM), and.
(d) introducing a therapeutically effective amount of the multipotent ATZ stem cells within the mixed population into the damaged or dysfunctional tissue in the subject.

18. A method of treating damaged or dysfunctional mesodermal tissue in a subject, comprising:
(a) obtaining a sample of ATZ cells that are autologous or allogeneic to the subject;
(b) isolating a mixed population of ATZ cells comprising multipotent ATZ stem cells and mesodermal progenitor ATZ cells from the sample;
(c) expanding the multipotent ATZ stem cells within the mixed population by culturing the mixed population in a minimal growth medium (MGM),
(d) culturing the mixed population in a mesodermal lineage-specific medium (LSM) to promote at least partial differentiation of multipotent ATZ stem cells within the mixed population to produce an expanded population of mesodermal progenitor ATZ cells within the mixed population; and
(e) introducing a therapeutically effective amount of the multipotent ATZ stem cells and mesodermal progenitor ATZ cells into the damaged or dysfunctional tissue in the subject.

19. The method of any one of claims 17-18, wherein:
the multipotent ATZ stem cells do not express significant levels of CD45.

20. The method of any one of claims 17-19, wherein:
the multipotent ATZ stem cells express markers CD34, CD117 and CD184.

21. The method of any one of claims 17-20, wherein:
wherein the step of expanding the multipotent ATZ stem cells comprises replating the ATZ cells through at least 5 passages in MGM.

22. The method of claim 18, wherein:
the damaged or dysfunctional mesodermal tissue is cardiac muscle tissue and the LSM comprises a cardiomyocyte-specific LSM.

23. The method of claim 22, wherein:
wherein the LSM promotes differentiation of multipotent ATZ stem cells to produce a population of ATZ stem cell-derived cardiomyocyte organoid cells.

24. The method of claim 18, wherein:
the damaged or dysfunctional mesodermal tissue is skeletal muscle tissue and the LSM comprises a skeletal muscle-specific LSM.

25. The method of claim 24, wherein:
wherein the LSM promotes differentiation of multipotent ATZ stem cells to produce a population of ATZ stem cell-derived skeletal muscle organoid cells.

26. The method of claim 18, wherein:
the damaged or dysfunctional mesodermal tissue is cartilage tissue and the LSM comprises an chondrocyte-specific LSM.

27. The method of claim 26, wherein:
wherein the LSM promotes differentiation of multipotent ATZ stem cells to produce a population of ATZ stem cell-derived chondrocyte organoid cells.

28. The method of claim 18, wherein:
the damaged or dysfunctional mesodermal tissue is kidney tissue and the LSM comprises a kidney-specific LSM.

29. The method of claim 28, wherein:
wherein the LSM promotes differentiation of multipotent ATZ stem cells to produce a population of ATZ stem cell-derived kidney organoid cells.

30. A cellular composition, comprising:
a mixed population of ATZ cells comprising multipotent ATZ stem cells and mesodermal progenitor ATZ cells in a pharmaceutically-acceptable carrier or exogenous biocompatible scaffold.

31. The cellular composition of claim 30, wherein:
the multipotent ATZ stem cells comprise at least 5% of the total ATZ cells.

32. The cellular composition of claim 30 or claim 31, wherein:
the multipotent ATZ stem cells do not express significant levels of CD45.

33. The cellular composition according to any of claims 30-32, for use as a medicament

34. A cellular composition for use in a method of treating damaged or dysfunctional mesodermal tissue, in a subject, wherein:
the composition comprises isolated ATZ cells and/or ATZ-derived organoid cells.

35. The cellular composition for the use of claim 34, wherein:
the isolated ATZ cells and/or ATZ-organoid cells comprise ATZ crypt cells, such as ATZ multipotent stem cells.

36. The cellular composition for the use of claim 34 or claim 35, wherein:
the isolated ATZ cells and/or ATZ-organoid cells comprise mesodermal progenitor ATZ cells.

37. The cellular composition for the use of any one of claims 34-36, comprising:
a pharmaceutically-acceptable carrier or exogenous biocompatible scaffold.

38. The cellular composition for the use of any of claims 34-37, wherein the subject is a human.

39. The cellular composition for the use of any of claims 34-38, wherein the damaged or dysfunctional tissue is kidney tissue.

40. The cellular composition for the use of any of claims 34-38, wherein the damaged or dysfunctional tissue is heart tissue.

41. The cellular composition for the use of any of claims 34-38, wherein the damaged or dysfunctional tissue is striated muscle tissue.

42. The cellular composition for the use of any of claims 34-38, wherein the damaged or dysfunctional tissue is cartilage tissue.

43. The cellular composition for the use of any of claims 34-42, wherein the isolated ATZ cells and/or ATZ-derived organoid cells are allogenic to the subject.

44. The cellular composition for the use of any of claims 34-43, wherein the isolated ATZ cells and/or ATZ-derived organoid cells are autologous to the subject.
